SEQUENCE LISTING (4 sequences)

SEQ ID NO: 1 - DNA, 20 bp, Artificial Sequence
  Primer
acaaggatgacaagcacagc

SEQ ID NO: 2 - DNA, 20 bp, Artificial Sequence
  Primer
gctgtgcttgtcatccttgt

SEQ ID NO: 3 - DNA, 40 bp, Artificial Sequence
  Primer
gtcggtcaagaacgagcactcaagagcctcagtcagacga

SEQ ID NO: 4 - DNA, 40 bp, Artificial Sequence
  Primer
tcgtctgactgaggctcttgagtgctcgttcttgaccgac

(19) United States
(12) Patent Application Publication
(10) Pub. No.: US 2016/0348150 A1
(43) Pub. Date: Dec. 1, 2016

(54) ELECTROCHEMICAL METHOD FOR DENATURING OR HYBRIDISING NUCLEIC ACID MOLECULES

(71) Applicant: THE UNIVERSITY COURT OF THE UNIVERSITY OF EDINBURGH, Edinburgh (GB)

(72) Inventors: Till Bachmann, Edinburgh (GB); Andrew Mount, Edinburgh (GB); Jason Crain, Edinburgh (GB); Schulze Holger, Edinburgh (GB)

(21) Appl. No.: 14/442,243

(22) PCT Filed: Nov. 12, 2013
(86) PCT No.: PCT/GB2013/052975

§ 371 Date: May 12, 2015

(30) Foreign Application Priority Data
Nov. 12, 2012 (GB) 1220282.6

(51) Int. Cl. C12Q 1/68

(52) U.S. Cl. CPC C12Q 1/686

(57) ABSTRACT

Denaturation and hybridisation of double-stranded DNA is a crucial reaction in many biological processes, such as DNA replication. DNA denaturation and hybridisation can be controlled by e.g. temperature, altering the pH and ionic strength and different chemical agents. This reversible reaction also plays a role in many diagnostic-based methods and applications such as any nucleic acid amplification method. The present invention provides alternate means to control denaturation and hybridisation of nucleic acids comprising contacting a nucleic acid molecule with a compound capable of interacting with a nucleic acid molecule and altering the state or a property of the compound to achieve denaturation or hybridisation of the nucleic acid molecule.

### FIELD OF THE INVENTION

The present invention provides methods for controlling interactions between nucleic acids, nucleic acid molecules and nucleic acid strands. For example, the invention provides methods for denaturing and hybridising nucleic acid molecules/strands. The invention finds particular application as a method for achieving the cyclic denaturation and hybridisation of nucleic acid molecules/strands.

### BACKGROUND OF THE INVENTION

Denaturation and hybridisation of double-stranded DNA (dsDNA) is a crucial reaction in many biological processes, such as DNA replication. These processes involve the breakage and rebuilding of hydrogen bonds (H-bonds), as well as destabilisation and stabilisation of π-π bonds and van der Waal interactions which are involved in base stacking.¹⁻⁴ DNA denaturation and hybridisation can be controlled by e.g. temperature, altering the pH and ionic strength, and different chemical agents, such as formamide and urea which either break or interfere with the H-bonds.⁵⁻⁹ This reversible reaction also plays an integral role in many diagnostic-based methods and applications, such as biosensors¹⁰ and microarrays¹¹⁻¹² or any nucleic acid amplification method (e.g. the polymerase chain reaction or ligase chain reaction)¹³⁻¹⁵.

An interest in making diagnostics cheaper, faster, more accurate and user-friendly has generated a focus on integration of miniaturised and fully automated laboratory operations, such as nucleic acid amplification techniques, into lab-on-a-chip devices or micro total analysis systems (μTAS).^{14, 16-18} This has aided the possibility of portable analysis, i.e. point-of-care testing, where the concept is to bring molecular diagnostics testing conveniently to the site or near the patient leading to a cost-effective approach as results can be obtained faster. Ideally, these systems should be fully automated, simplified with only a few analytical steps and easy to use.¹⁹⁻²⁰ Electrochemistry-based sensing offer high sensitivity, fast response, simpler operations and low cost. It also simplifies miniaturisation and integration of automated laboratory operations into ρTAS.²¹⁻²² Thus, being able to control denaturation and hybridisation of nucleic acids electrochemically, instead of thermally, may be valuable in advancing areas such as portable nucleic acid-based diagnostics where assay simplification is crucial.

Wang and co-workers investigated electrochemically-driven pH cycling leading to DNA denaturation and renaturation in an electrolytic cell with two chambers, each containing a working electrode (bifacial hydrogen-permeable palladium thin foil) and an auxiliary electrode. Using 290 bp DNA fragments, cyclic denaturation and renaturation was obtained by applying a polarization current and zero current to alter and maintain the pH between 5.2 and 11.4, with each cycle lasting 300 s. The change was monitored spectrophotometrically at 260 nm and a hyperchromic shift of ˜47% was observed. Gel electrophoresis confirmed no severe DNA degradation.⁸ Others have investigated photoregulation of DNA denaturation and hybridisation by introducing azobenzene units into short 8 bp long DNA oligonucleotides. Upon irradiation of UV light, the azobenzene unit underwent structural change from trans to cis, thereby lowering the T_{M} of the DNA below room temperature and thus promoting denaturation. The structural changes reverse upon irradiation with visible light facilitating hybridisation.²³⁻²⁴

Cheng et al³³ investigated the effects of the metabolic process of DM on fish sperm DNA in vitro. Here, daunomycin (DM) was reduced electrochemically on a graphite plate and the effects were monitored spectrophotometrically. Nevertheless, the authors concluded that DM damaged the DNA by fragmentation due to free radical generation upon the reduction.

Despite elegant solutions to replace thermal control of DNA denaturation and hybridisation, some still require a long cycle time for the reversible reaction to occur or require covalent modification of the DNA bases. Hence, there is still a need for a fast, electrochemically controlled solution.

### SUMMARY OF THE INVENTION

The present invention is based on the finding that interactions between nucleic acid molecules or strands, may be controlled by altering or modulating a property or the state of a compound interacting with the nucleic acid or nucleic acid molecule. One of skill will appreciate that an interaction between nucleic acid molecules may be a “hybridisation” type interaction where one nucleic acid molecule (or strand) is bound to a complementary nucleic acid molecule (or strand). Accordingly, the present invention relates to methods which may be used to modulate nucleic acid molecule hybridisation.

In a first aspect, the present invention provides a method of denaturing or hybridising nucleic acid molecules, said method comprising the steps of:

contacting a nucleic acid molecule with a compound capable of interacting with a nucleic acid molecule; and

altering the state or a property of the compound to achieve denaturation or hybridisation of the nucleic acid molecule.

For simplicity, the terms “nucleic acid”, “nucleic acid molecule” and “nucleic acid strand” shall be collected together under the term “nucleic acid molecule”. References to nucleic acid molecules may encompass double or single stranded nucleic acid molecules. One of skill will appreciate that a nucleic acid molecule, may comprise a sugar phosphate backbone and one or more nucleotides to form a nucleic acid strand. The term “nucleic acid molecule” may be applied to single and/or or double stranded deoxyribonucleic acid (DNA), ribonucleic acid (RNA), nucleic acid which represents a combination of both DNA and RNA and synthetic nucleic acids. One of skill will appreciate that synthetic nucleic acids may include one or more synthetic (non-naturally occurring) nucleotides. All forms of nucleic acid are to be understood as encompassed within the term “nucleic acid molecule”. For example, the term “nucleic acid molecule” may embrace oligonucleotides, nucleic acid primers and other nucleic acid probes. One of skill will appreciate that oligonucleotides, primers and probes may be hybridised to (and subsequently dissociated from) other types of nucleic acid molecule—for example any form of template or target nucleic acid sequence or (isolated, purified or cloned) genomic nucleic acid, cDNA or fragments thereof. The term “nucleic acid” may further include, for example “amplicon” type nucleic acids—that is nucleic acid which is the product of some form of amplification process.

The methods provided by this invention may be exploited to control interactions between nucleic acid molecules or strands. One of skill will appreciate that a nucleic acid molecule or strand may, through hydrogen bonding between bases, hybridise, bind, anneal to another (complementary) nucleic acid molecule or strand to form a duplex (double stranded) structure. These nucleic acid duplex (double stranded) structures may, under suitable conditions (namely conditions which bring about the breaking of the hydrogen bonds formed during the formation of duplex structures) de-nature, melt or dissociate into single strands.

In view of the above, the present invention may be exploited in order to achieve the denaturation, de-hybridisation, melting or dissociation of double stranded nucleic acid molecules. For brevity, the terms “denaturation”, “de-hybridisation”, “melting” and “dissociation” shall be collected together under the single term “denaturation”. During the denaturation process, a double stranded nucleic acid may become two separate, single strands of nucleic acid—in other words, denaturation of a double stranded nucleic acid molecule involves the dissociation of the two hybridised strands into two separate, single strands of nucleic acid.

The methods of this invention may also be used to hybridise, anneal or re-nature nucleic acid molecules. For brevity, the terms “hybridise”, “anneal” and “re-nature” shall be collected under the single term “hybridise”. The term “hybridise” may be used to describe the formation of a bond or association between two entities. As applied to nucleic acid, the term “hybridise” may encompass the renaturation or re-formation of a double stranded nucleic acid molecule from two or more single nucleic acid strands. Additionally, or alternatively, the term “hybridise” may encompass processes involving the formation of a bond or association between single strands of nucleic acid so as to form a double stranded nucleic acid molecule.

Accordingly, the methods provided by this invention may be used to denature a double stranded nucleic acid molecule or to hybridise a single nucleic acid strand to or with, another single nucleic acid strand. The methods may be exploited to achieve the formation of duplex nucleic acid molecules.

The methods may also be used to achieve continual or cyclic denaturation and hybridisation of nucleic acids; in such cases double stranded nucleic acid molecules may be sequentially and repeatedly denatured and re-natured and single stranded nucleic acid molecules may be sequentially and repeatedly hybridised (to form double stranded molecules) and denatured.

Without wishing to be bound by theory, the inventors have discovered that if a property or state of a compound interacting with a nucleic acid molecule is changed, the nucleic acid can either be denatured (if it is in double stranded form) or hybridised to another (single stranded) nucleic acid (if it is in single stranded form). The methods may also be exploited in order to achieve the continual or cyclic denaturation and/or renaturation of nucleic acid.

The methods provided by this invention may utilise one or more compounds capable of interacting with nucleic acid. A compound capable of interacting with a nucleic acid molecule may bind to, or otherwise associate with single or double stranded nucleic acid molecules through the formation of covalent bonds, non-covalent bonds, electrostatic interactions, van der Waals interactions and/or hydrogen bonds and the like. The compounds to be exploited in the methods described herein may be capable of interacting with double stranded nucleic acid molecules. In all the methods described herein, the compound may be contacted with nucleic acid under conditions (time, temperature, osmolarity and pressure) which favour or facilitate the formation of an interaction with nucleic acid.

Compounds suitable for use in this invention may interact or associate with nucleic acid molecules by intercalation. Compounds of this type may be referred to as nucleic acid intercalating compounds or “intercalators”. An intercalator compound may comprise a small organic and/or planar molecule. Suitable intercalator compounds may comprise aromatic moieties and may interact with nucleic acid (and in particular double stranded nucleic acid molecules) via electrostatic, covalent and/or non-covalent interactions and/or via van der Waals interactions and/or hydrogen bonds and the like. Without wishing to be bound by theory, an intercalator compound may interact with nucleic acid by insertion or location between the (stacked) nucleobases pairs. Side effects of intercalation may include, for example, complete or partial unwinding of the double helix structure, lengthening of the nucleic acid and/or increased rigidity. One of skill will appreciate that any increase in rigidity may occur as a consequence of interactions between side-groups and substituents of the intercalator compound and moieties of the nucleobases pairs, the nucleic acid back bone and/or the surface of the double helix. By spectrophotometric analysis, intercalation may be detected as a decrease in the absorbance of the intercalator and/or as a red-shift of the intercalator peak.

A change of property or state of any of the compounds described herein (such as, for example the intercalator compounds) may comprise a change in the oxidation status or redox state of the compound. For example, it may be possible to oxidise or reduce any of the compounds described herein using any suitable technique including, for example, chemical and/or electrochemical methods. Compounds which, under certain chemical and/or electrochemical conditions undergo a change in oxidation or redox status, may be referred to as “redox-active” compounds. As such, the present invention may extend to methods which exploit redox-active compounds capable of interacting with nucleic acid. In particular, the methods of this invention may exploit redoxactive compounds which are capable of interacting (by intercalation) with double stranded nucleic acid molecules.

A change in the redox-state of a compound may be induced by exposure or contact of the compound to or with a reducing agent or an oxidising agent. A reducing agent may be an electron donor and may thus be capable of donating electrons to a compound of this invention. An oxidising agent may be an agent which is capable of removing electrons from a compound of this invention. There are many suitable forms of reducing or oxidising agent which may be used to affect a change in the redox state of a compound provided by this invention and the particular choice of reducing/oxidising agent may depend on the nature of the compound capable of interacting with nucleic acid. Examples of reducing agents may include, for example sodium borohydride (NaBH₄) and/or ascorbic acid. Other suitable reducing and/or oxidising agents will be well known to one of skill in this field.

In view of the above, the present invention provides a method of denaturing or hybridising nucleic acid molecules, said method comprising the steps of:

contacting a nucleic acid molecule with a compound capable of interacting with nucleic acid; and

contacting the compound with a reducing or oxidising agent to achieve oxidation or reduction of the compound and denaturation or hybridisation of the nucleic acid molecule.

Without wishing to be bound by theory, the inventors hypothesise that upon alteration or modulation of the oxidation (or redox) state of a compound described herein, hydrogen bonding patterns between the compound (for example a nucleic acid intercalator compound) and the nucleic acid molecule, as well as hydrogen bonding patterns within the nucleic acid molecule itself, change causing the nucleic acid to denature (if it is double stranded) or hybridise (if it is single stranded). Additionally, or alternatively (and again without wishing to be bound by theory), it is suggested that to that under suitable conditions and upon alteration or modulation of the redox state of the (intercalator) compound, the hydration state changes which in turn affects the net charge of the molecule. It is possible that under these conditions, the (intercalator) compound may dissociate (de-intercalate) from the nucleic acid, de-stabilising it and bring about denaturation.

As such, by altering the redox-state of a compound capable of associating (perhaps by intercalation) with nucleic acid, it is possible to affect either the denaturation or renaturation of a nucleic acid molecule.

By way of example, an oxidised compound of this invention may interact with double stranded nucleic acid molecules. Without wishing to be bound by theory, if the redox state of a compound interacting with a double-stranded nucleic acid molecule is altered such that the compound becomes reduced, the double stranded nucleic acid molecule may be denatured (i.e. melted into single strands). Conversely, the inventors have observed that in a solution comprising single stranded nucleic acids and the reduced form of a compound capable of interacting with nucleic acid (for example double stranded nucleic acid), oxidation of the reduced from of that compound brings about hybridisation between the single stranded nucleic acid molecules (forming double stranded nucleic acid molecules). The oxidised form of the compound may then interact with the newly formed double stranded nucleic acid molecules. In view of the above, one of skill will appreciate that by continually cycling the oxidation status of a compound capable of interacting with nucleic acid between reduced and oxidised states in the presence of nucleic acid molecules, it is possible to achieve the continual or cyclic denaturation and hybridisation of the nucleic acid molecules.

A property or state of a compound may be altered or modulated by electrochemical means and therefore, compounds for use in this invention may be electroactive compounds which are capable of interacting with nucleic acid. An electroactive compound of this invention may interact with nucleic acid (for example double-stranded nucleic acid) when in an oxidised form.

As such, this invention may provide a method of denaturing or hybridising nucleic acid molecules, said method comprising the steps of:

contacting a nucleic acid molecule with an electroactive compound capable of interacting with nucleic acid; and

electrochemically altering the state or a property of the compound to achieve denaturation or hybridisation of the nucleic acid molecule.

One of skill will appreciate that an electroactive compound may be a compound which when exposed to an electric current or potential or when stimulated with an electric current or potential, undergoes some form of property and/or state change. Suitable electroactive compounds may include those which, upon exposure to or stimulation with an electric current or potential, undergo a change in oxidation status. For example, electroactive compounds for use in this invention may exhibit a change in redox state (i.e. they may undergo reduction or oxidation) when exposed to or stimulated with an electric current or potential.

The methods provided by this invention may exploit electroactive (or electro-redoxactive) intercalator compounds. In addition to being able to interact with, for example, double-stranded nucleic acid through intercalation, when exposed to or stimulated with an electric current or potential, an electroactive intercalator compound may exhibit a change in property or state. When exposed to or stimulated with an electric current or potential, an electroactive intercalator compound may undergo a change in oxidation state. For example, exposure to or stimulation with an electric current or potential may alter the redox state of the electroactive intercalator such that the electroactive intercalator may become reduced or oxidised.

Accordingly, the present invention provides a method of denaturing or hybridising nucleic acid molecules, said method comprising the steps of:

contacting a nucleic acid molecule with an electroactive compound capable of interacting with nucleic acid; and

exposing the electroactive compound to an electric current or potential, or stimulating the electroactive compound with an electric current or potential so as to alter the oxidation status of the electroactive compound;

wherein alteration of the oxidation status of the electroactive compound affects denaturation or hybridisation of the nucleic acid molecule.

Moreover, the present invention provides a method of denaturing or hybridising nucleic acid molecules, said method comprising the steps of:

contacting a nucleic acid molecule with an electroactive intercalator compound; and

exposing the electroactive intercalator compound to an electric current or potential or stimulating the electroactive intercalator compound with an electric current or field so as to alter the oxidation status of said compound;

wherein alteration of the oxidation status of the electroactive intercalator compound affects denaturation or hybridisation of the nucleic acid molecule.

The electroactive intercalator compound may be contacted with the nucleic acid molecule under conditions which permit intercalation of the compound with a double stranded nucleic acid molecule.

Suitable electroactive intercalator compounds may include, for example, methylene blue (MB), anthraquinone (and its electroactive derivatives), metal complexes (including, for example, Fe(phen)₃²⁺ and Ru(phen)₃²⁺), 9-hydroxyellipticine and anthracycline compounds such as, for example, Adriamycin and daunorubicin (daunomycin) and electroactive derivatives thereof. Other suitable electroactive compounds are disclosed in Drummond et al (2003), Luo and Hsing (2009), Erdem et al (2001), Fang et al (2009), Yeung et al (2008) and Defever et al (2011).

An exemplary electroactive intercalator compound for use in the methods described herein is the intercalator compound daunorubicin ([(8S,10S)-8-Acetyl-10-[(3-amino-2, 3,6-trideoxy-α-L-lyxo-hexopyransoyl)oxy]-7,8,9,10-tetrahydro-6,8,11-trihydroxy-1-methoxy-5,12-naphthacenedione hydrochloride; otherwise known as daunomycin, daunomycin cerubidine and “DM”). As with all the electroactive/nucleic acid interacting compounds described herein, the terms “daunorubicin” or “daunomycin” extends to all derivatives which exhibit electroactive properties and an ability to interact with nucleic acid. In this regard, the invention extends to methods which exploit daunorubicin derivatives which, when exposed to or stimulated with, an electric current or potential, exhibit a change in some property or state. For example, suitable daunorubicin derivatives may, when exposed to or stimulated with an electric current or field, exhibit (or undergo) a change in oxidation state. By way of example, suitable daunorubicin derivatives may include any of those recited in U.S. Pat. No. 4,191,756 and U.S. Pat. No. 4,256,632.

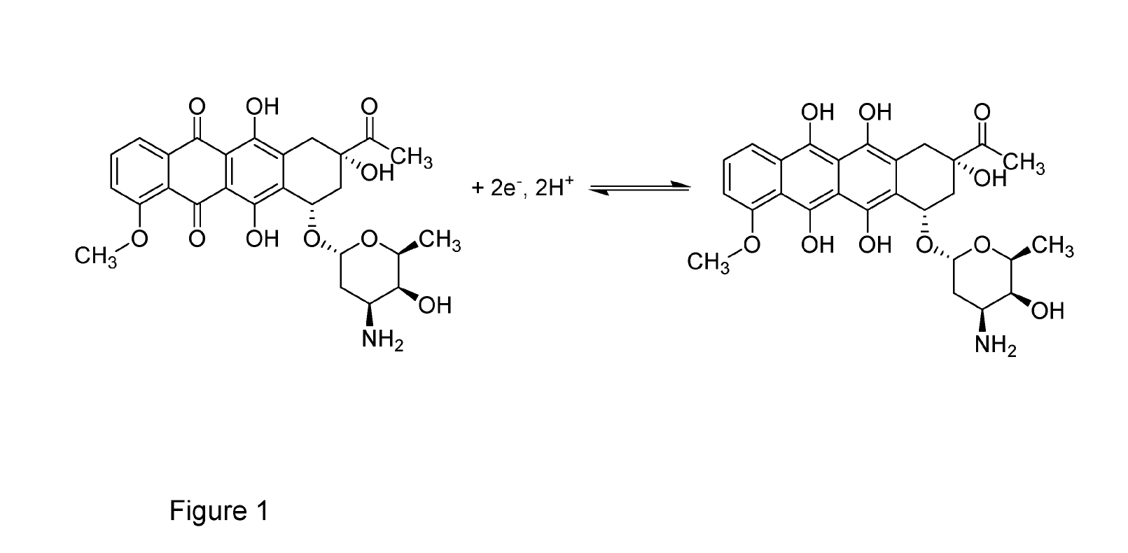

Daunorubicin consists of an electroactive aglycone unit, which intercalates into DNA, and one sugar ring, which binds in the minor groove of DNA. FIG. 1 shows further detail. Crystallography studies by Quigley et al showed that one DM binds per every three base pairs (bp) and it is stabilised by three H-bonds as well as several van der Waals interactions.³¹ The oxidised form of DM has been found to increase the melting temperature (T_{M}) of DNA by up to 30° C.³²

Certain compounds which are capable of interacting with nucleic acid and which find application in the present invention may exist in a naturally oxidised or reduced state. Compounds of this type may be added directly to nucleic acid molecules to affect hybridisation and/or denaturation as required. Upon alteration of the naturally oxidised or reduced state of the compound (to the other of a reduced or oxidised state), the nucleic acid molecules may be caused to denature or hybridise. Thereafter continual hybridisation and/or denaturation of nucleic acid molecules may be affected by, for example, subjecting or exposing the nucleic acid molecule/compound complexes to any of the means (agent or electrochemical based) described herein which affect a change in oxidation (redox) state of the compound. By way of example, daunorubicin exists naturally in an oxidised state and therefor addition of naturally oxidised daunorubicin to nucleic acid molecules may trigger or induce hybridisation thereof. In its reduced form, daunorubicin may trigger or induce the denaturation of hybridised nucleic acid molecules.

Thus, the present invention provides a method of denaturing or hybridising nucleic acid molecules, said method comprising the steps of:

contacting a nucleic acid molecule with daunorubicin and/or an electroactive and intercalating derivative thereof; and

exposing the daunorubicin or derivative thereof to an electric current or potential or stimulating the daunorubicin or derivative thereof with, an electric current or potential;

wherein exposing the daunorubicin or derivative thereof to an electric current or potential or stimulating the daunorubicin or derivative thereof with an electric current or potential, modulates the oxidation status of the daunorubicin or derivative thereof resulting in denaturation or hybridisation of the nucleic acid molecule.

One of skill will appreciate that, an electric potential may be referred to as a “negative potential” or a “positive potential”—the terms “negative” and “positive” being used to describe the potential versus (or relative to) a reference electrode.

When exposed to or contacted with a negative electric potential, certain electroactive compounds may adopt a reduced or oxidised state. Conversely, when exposed to a positive electric potential, certain electroactive compounds may adopt an oxidised or reduced state. An electric potential which brings about the reduction of a compound may be known as a “reduction potential” (Eₐ), whereas an electric potential which brings about the oxidation of a compound may be known as an “oxidation potential” (E_{c}).

An electroactive compound for use in this invention may adopt a reduced state when exposed to or stimulated with a reduction potential. Conversely, when exposed to or stimulated with an oxidation potential, an electroactive compound for use in this invention may adopt an oxidised state.

The oxidation potential of (E_{c}) of daunorubicin is approximately in the region of −0.5V to +0.8 V vs Ag/AgCl. The oxidation potential (E_{c}) of daunorubicin may be approximately −0.41V vs Ag/AgCl. As such, when exposed to, or stimulated with, a potential of about −0.41V, daunorubicin may adopt an oxidised state.

The reduction potential (Eₐ) of daunorubicin is approximately in the region of −1.3V to −0.3 V vs Ag/AgCl. The reduction potential (Eₐ) of daunorubicin may be approximately −0.61V vs Ag/AgCl. As such, when exposed to, or stimulated with, a potential of about −0.61V, daunorubicin, may adopt a reduced state.

In its oxidised state, daunorubicin may intercalate with double stranded nucleic acid molecules. Upon altering the redox state of oxidised and intercalated daunorubicin, the nucleic acid molecule (into which oxidised daunorubicin is intercalated) may be denatured. By further altering the redox state of the now reduced daunorubicin, denatured (i.e. single stranded) nucleic acid molecules may be re-hybridised to form double-stranded nucleic acid.

The methods described herein may be conducted at a temperature which facilitates the denaturation and/or hybridisation of nucleic acid molecules. Suitable temperatures may be referred to as “working” temperatures. A suitable working temperature may further facilitate the interaction of the various compounds (for example the electroactive intercalating compounds) with nucleic acid. A working temperature may be determined using melting curve analysis. Melting curve analysis may exploit one or more nucleic acids in, for example, solution. The nucleic acid molecules may comprise a homogeneous or heterogeneous population of nucleic acids. The nucleic acids may be labelled, for example fluorescently labelled. Melting curve analysis to establish a working temperature for any given compound capable of interacting with nucleic acid (for example an electroactive intercalator compound of this invention) may comprise determining the melting temperature (Tₘ) of a nucleic acid molecule (a) in the presence of an oxidised compound capable of interacting with nucleic acid and (b) in the presence of a reduced form of that compound—optionally, the analysis may further involve determining the melting temperature (Tₘ) of the nucleic acid molecule in absence of oxidised or reduced forms of the compound capable of interacting with nucleic acid. The working temperature is determined from the melting curves as the temperature at which 100% of the nucleic acid molecule is hybridised when the compounds of the present invention are in one oxidation status (for example in an oxidised or reduced state) and 100% of the nucleic acid molecule is denatured when the compounds of the present invention are in another oxidation status (for example reduced or oxidised state). For example, the working temperature may fall between the end-point of the melting curve of a nucleic acid molecule in the presence of an oxidised compound of the invention, and the end-point of the melting curve of that nucleic acid molecule in the presence of a reduced form of the compound. The skilled man will recognise that a compound capable of interacting with nucleic acid may trigger the hybridisation of nucleic acid when it is in its oxidised form and it may trigger the denaturation of nucleic acid when it is in its reduced form. A working temperature may be any temperature within a range of about ±5° C. of a temperature calculated in accordance with the aforementioned method. Thus, the methods of the invention may comprise a step (for example an initial step) in which a suitable working temperature is calculated. When determining a working temperature, the compound capable of interacting with nucleic acid may be present in an oxidised or reduced form. For example, where a method for denaturing or hybridising nucleic acid exploits the intercalator compound daunorubicin, the working temperature may be the temperature at which 100% of the DNA, in the presence of reduced daunorubicin, is in a denatured state and 100% of the DNA in the presence of oxidised daunorubicin is in a hybridised state. A working temperature may be calculated by analysing DNA melting at a range of temperatures in the presence of a compound capable of interacting with nucleic acid—for example reduced and/or oxidised electroactive intercalator compounds such as, for example, daunorubicin.

In methods which exploit, for example, daunorubicin, the working temperature may be anywhere between about 20-25° C. and about 99° C. (the disclosed range starting and ending at any two temperatures within this range). For example, the working temperature may be in the range of about 20° C. to about 99° C., 20° C. to 25° C., 30° C. to 90° C., 40° C. to 80° C., 50° C. to 70° C. In a preferred embodiment the working temperature may be about 75° C. In another embodiment the working temperature may be room temperature, about 20° C. to 25° C.

The precise working temperature may vary depending on, for example, the length and size of the nucleic acid molecules to be hybridised and/or denatured.

In view of the above, any of the methods described herein may comprise a step in which a nucleic acid molecule to be denatured or hybridised, is contacted with a compound capable of interacting with nucleic acid at a suitable working temperature. The redox state of the compound may then be chemically or electrochemically altered/modulated so as to bring about nucleic acid molecule denaturation or hybridisation as required.

The methods described herein may further comprise the use of conditions of low, medium or high salt concentrations. Without wishing to be bound to theory, increasing salt concentrations may help diffuse negative repulsions between the moieties within the backbone of a nucleic acid molecule. This stabilisation may lead to an increase in the melting temperature of the nucleic acid molecule. For example a compound capable of interacting with nucleic acid may be contacted with a nucleic acid molecule (which nucleic acid molecule is to be hybridised to another nucleic acid molecule or denatured) under conditions which comprise a salt (standard saline sodium citrate) concentration of about 0.01-6×SSC. It should be understood that the range 0.01-6.00×SSC includes every concentration value between these end points. For example, the methods described herein may utilise buffers having a relatively low salt concentration of about 0.036×SSC, a medium salt concentration of about 0.04-0.08×SSC and a relatively high salt concentration of about 0.09-0.10×SCC to about 0.15-0.30×SCC.

The methods described herein may further be conducted at a predetermined pH. Without wishing to be bound to theory, the increase of pH may destabilise double stranded DNA, which can lead to a decrease in DNA's melting temperature. For example, the methods of this invention may be conducted at about pH 5, pH 6, pH 7, pH 8 or pH 9. A method of this invention may be conducted at about pH 7.

The methods provided by this invention may find particular application in protocols which require the cyclic denaturation and hybridisation of nucleic acids. For example, the methods described herein may be exploited in protocols for amplifying and/or detecting nucleic acids.

Methods of amplifying nucleic acid may comprise the ligase and/or polymerase chain reaction (LCR or PCR: or variations thereof) where primer sequences are used to amplify specific sections or sequences of nucleic acid. Such methods may require an initial double-stranded nucleic acid sample to be denatured (i.e. dissociated into single strands) and this may be achieved through the use of thermal energy. Moreover, as nucleic acid amplification protocol (PCR) proceeds, subsequent steps involving primers and template nucleic acid sequences may further require the use of procedures which affect the denaturation and/or nucleic acid sequences to be denatured (i.e. dissociated) or hybridised. A double-stranded nucleic acid (either a template sequence or a primer:template complex (duplex)), may be denatured by exposing the nucleic acid to a first temperature and re-natured (or hybridised) by exposing it to a second lower temperature. More detailed information on different methods for amplifying nucleic acid, including for example polymerase chain reaction based methods, may be found in Molecular Cloning: A Laboratory Manual (Fourth Edition), CSHP, Green, Hughes Sambrook & MacCallum. Other information may be derived from, for example U.S. Pat. No. 4,965,188. The entire content of these references is incorporated herein by reference.

The present invention provides an alternative means for achieving nucleic acid (for example primer and template (or amplicon) nucleic acid) denaturation or hybridisation as might be required in, for example a nucleic acid amplification protocol such as PCR. Moreover, the present invention dispenses with the need to use large and costly thermocycling equipment to achieve cyclic nucleic acid denaturation/hybridisation.

Thus in a second aspect, the present invention provides a method of achieving the denaturation or hybridisation of nucleic acid molecules in a nucleic acid amplification process, said method comprising:

during an amplification process, contacting a nucleic acid molecule with a compound capable of interacting with nucleic acid as described herein; and

altering a property or state of the compound;

wherein, by altering a property or state of the compound, the nucleic acid molecule denatures or hybridises.

It should be understood that the definitions of features of the first aspect of this invention apply to the equivalent terms of the second aspect of this invention. Accordingly, the compound may be a compound capable of interacting with nucleic acid by intercalation. Moreover, the compound may be an electroactive compound.

The property or state to be altered may be the oxidation or redox state of the compound and depending upon the nature of the compound, the redox state may be altered by chemical and/or electrochemical means as described above. Where the compound is an electroactive intercalator compound, the redox state of the compound may be altered by exposing the compound to, or stimulating the compound with, an electric current or potential.

The present invention may also provide a method of achieving the denaturation or hybridisation of nucleic acid molecules in a nucleic acid amplification process, said method comprising:

providing a template nucleic acid sequence to be amplified;

contacting the template nucleic acid sequence to be amplified with one or more primer sequences and a compound capable of interacting with nucleic acid;

altering a property or state of the compound capable of interacting with nucleic acid so as to achieve hybridisation or denaturation of the template nucleic acid sequence and the one or more primers.

To achieve amplification, it may be necessary to add nucleosides (dNTPs) and/or to repeat the method steps at least once.

The template nucleic acid molecule may comprise genomic or cloned nucleic acid or a fragment thereof.

The invention may provide a polymerase chain reaction amplification method, said method comprising:

providing a template nucleic acid sequence to be amplified;

contacting the template nucleic acid sequence to be amplified with one or more primer sequences and a compound capable of interacting with nucleic acid;

altering a property or state of the compound capable of interacting with nucleic acid so as to achieve hybridisation or denaturation of the template nucleic acid sequence and the one or more primers.

As stated, the method may further comprise contacting the template nucleic acid with nucleoside (triphosphate) molecules (dNTPs). Moreover, to achieve amplification, it may be necessary to repeat the method steps at least once. The template nucleic acid molecule may comprise genomic or cloned nucleic acid or a fragment thereof.

The inventors have noted that in some cases, compounds capable of interacting with nucleic acid for use in this invention may further interact with electrodes employed in electrochemical methods. For example, compounds which are capable of interacting with nucleic acid (for example the electroactive intercalator compounds described herein) may become adsorbed to (or otherwise bound to, associated with or coated on) electrodes used in electrochemical methods. It will be appreciated that these types of interaction may (adversely) affect the properties and/or performance of the electrodes. Moreover, as a consequence of interactions between compounds (capable of interacting with nucleic acids) used in this invention and the electrodes of an apparatus, the amount of nucleic acid hybridisation and/or denaturation (and amount of nucleic acid amplification) may be reduced as compared to the amount of hybridisation and/or denaturation (or amplification) which occurs in a system in which one or more of the electrodes have been treated in some way so as to reduce the occurrence of interactions between compounds (capable of interacting with nucleic acid) and the electrodes. Without wishing to be bound by theory, the inventors suggest that enzymes (for example DNA polymerase) may also be susceptible to interaction (perhaps adsorption or binding to or coating on or association with) an electrode.

As such, to (substantially) reduce, avoid, eliminate or modulate interactions between the electrodes of an apparatus for use in this invention and one or more of the components of the methods described herein, the electrodes for use maybe treated with a coating substrate. Application of a coating substrate to one or more of the electrodes may substantially reduce the incidence of interactions between, for example, compounds which are capable of interacting with nucleic acid and the electrodes themselves. It should be understood that the phrase “components of the methods described herein” may refer not only to, for example, the electroactive/intercalating compounds capable of interacting with nucleic acid, but also nucleic acids (including primers, amplicons, probes and template sequences), enzymes (for example DNA polymerase), dNTPs, buffers and divalent ions.

A coating substrate may be applied to an electrode as one or more layers. The coating substrate may be applied to all or part of an electrode. An electrode may be treated with a single type of coating substrate or two or more different coating substrates.

A coating substrate may comprise, consist essentially of or consist of a proteinaceous material. For example, a coating substrate may comprise, consist essentially of or consist of, albumin, for example bovine serum albumin (BSA).

A coating substrate may be applied to an electrode by, for example, screen-printing.

One of skill will appreciate that an electrode may comprise any suitable material including, for example carbon, indium tin oxide (ITO) and/or metals such as, for example platinum and/or gold. All electrodes suitable for use in a method of this invention may be subjected to pre-treatment with a coating substrate so as to prevent unwanted interactions with components for use in the method of this invention; which interactions may adversely affect nucleic acid hybridisation and/or denaturation processes.

Further information regarding the coating of electrodes may be found in U.S. Pat. No. 3,897,326 the entire content of which is incorporated herein by reference.

In view of the above, any of the electrochemical based methods described herein, including for example, the electrochemical based nucleic acid amplification protocols of this invention, may comprise the use of electrodes which have been treated with some form of coating substrate. Moreover, the methods may comprise a step in which the electrodes of an apparatus for use in an electrochemical based method of achieving nucleic acid hybridisation and/or denaturation (including for example, an electrochemical based nucleic acid amplification technique) are, prior to use, coated with a coating substrate.

In a further aspect, the present invention provides a kit, for hybridising and/or denaturing nucleic acid, said kit comprising one or more compounds capable of interacting with nucleic acid and/or a device capable of generating an electric current and/or field with one or more further components selected from the group consisting of:

apparatus for use in an electrochemical method;

coating substrates (for coating electrodes as described above);

buffers, components, solutions, reagents and/or enzymes;

receptacles and/or tools for manipulating solutions, reagents and the like.

Optionally, the kit provided by the third aspect of this invention may comprise instructions for use

The kit may be a kit for use in a method of amplifying or detecting nucleic acid. For example, the kit may be a polymerase chain reaction (PCR) kit and may additionally comprise, for example, one or more components selected from the group consisting of:

(a) one or more PCR components selected from the group consisting of:
(i) dNTPs;
(ii) primers;
(iii) nucleic acid (for example DNA) polymerase;
(iv) buffer solution; and
(v) divalent ions (for example Mg²⁺ or Mn²⁺);

(b) one or more electroactive compounds as described herein; and

(c) apparatus for use in an electrochemical method.

Optionally, any of the kits described herein may comprise instructions for use.

The inventors have noted that the compounds capable of interacting with nucleic acid molecules, in particular the electroactive intercalator compounds described herein may interfere with intercalator based nucleic acid detection methods. As such, nucleic acid molecules subjected to any of the methods described herein, may be best detected by non-intercalator detection methods. For example, spectrophotometry based methods may be used to detect and/or quantify nucleic acid that has been amplified and/or hybridised/denatured using a method of this invention. Additionally or alternatively, denaturing electrophoresis techniques may be used to rid a nucleic acid molecule of a compound interacting therewith (for example an electroactive intercalator compound) prior to attempting nucleic acid detection. Thus, nucleic acid subjected to any method of this invention may be detected using a non-intercalator based detection method.

In addition to the above, the inventors have also noted that by altering the state or a property of a compound interacting with a nucleic acid molecule (for example, a compound intercalated with a nucleic acid molecule), it is possible to modulate the melting temperature of the nucleic acid molecule.

As such, one aspect of this invention provides a method of altering the melting temperature of a nucleic acid molecule, said method comprising contacting a nucleic acid molecule with a compound capable of interacting with nucleic acid (as defined herein) and, altering a state or property of the compound so as to modulate the melting temperature of the nucleic acid molecule. It should be understood that the electrochemical and/or chemical methods described herein and used to alter a state or property (for example the redox or oxidation state) of the compound, apply to the methods described in this aspect of the invention.

The presence of an oxidised intercalator such as oxidised daunorubicin, may increase the melting temperature of a nucleic acid molecule. Conversely, the presence of a reduced intercalator such as reduced daunorubicin may also increase the melting temperature of the nucleic acid molecule but by less than oxidised daunorubicin.

### DETAILED DESCRIPTION OF THE INVENTION

The present invention will now be described with reference to the figures which show:

FIG. 1: Scheme showing the oxidation and reduction of daunorubicin (daunomycin, DM).

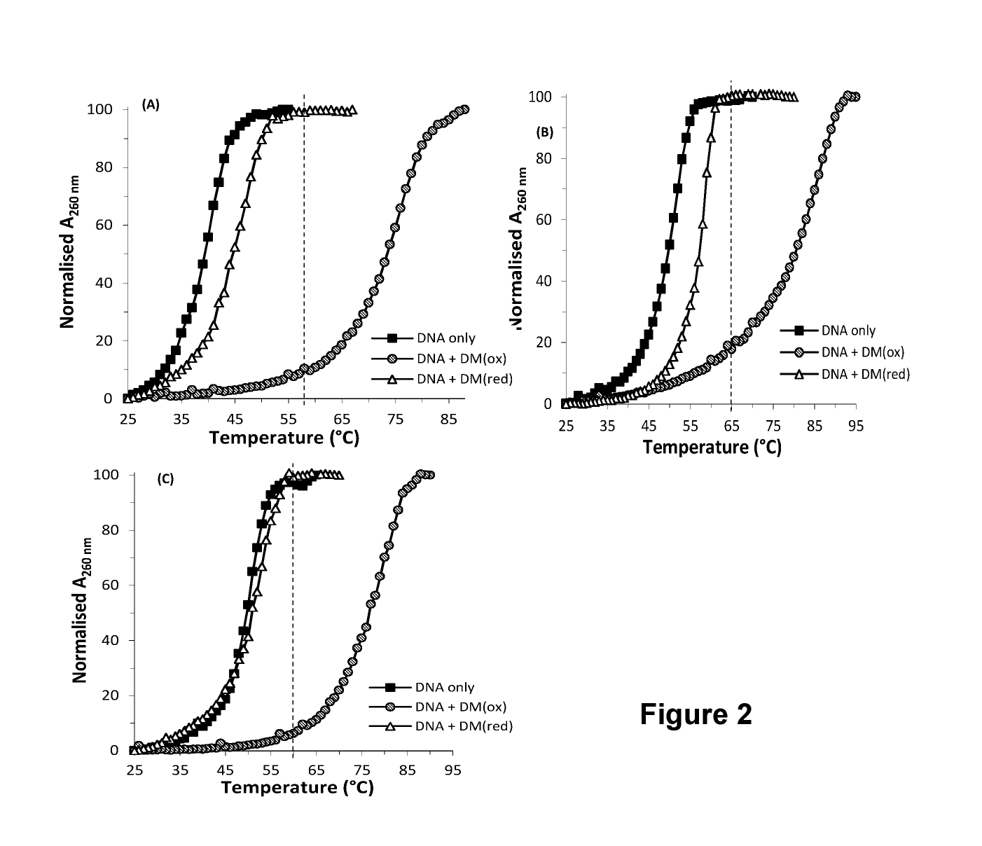

FIG. 2: DNA melting curves recorded at 1° C./min for 200 μM 20 mer or 40 mer DNA in presence and absence of 100 μM DM (molar ratio 2:1). Reduced daunorubicin (DM) is obtained chemically using NaBH₄ in excess (5 mM). (A) 20 mer at 0.036×SSC, (B) 40 mer at 0.036×SSC and (C) 20 mer at 0.1×SSC. Unlike DM_{red}, DMₒₓ increases T_{M} of DNA in all three conditions. Dashed lines represent optimal working temperatures in electrochemical switch experiments.

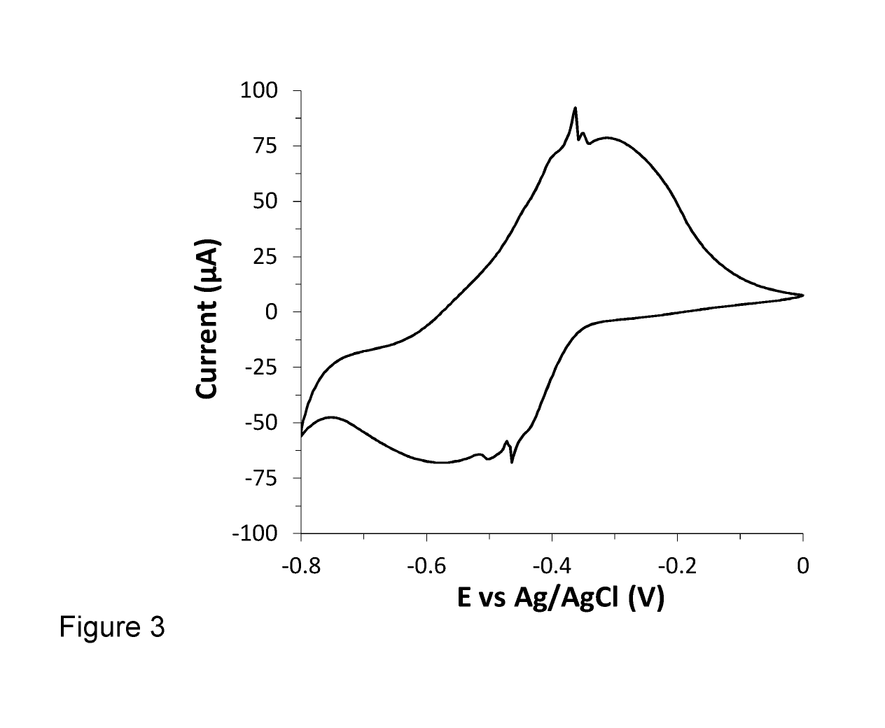

FIG. 3: Electrochemical characterisation of 100 μM DM in 0.036×SSC with cyclic voltammetry. 10^{th} CV scan at room temperature, recorded at 10 mV/s, where E_{c}=−0.41 V and Eₐ=−0.61 V.

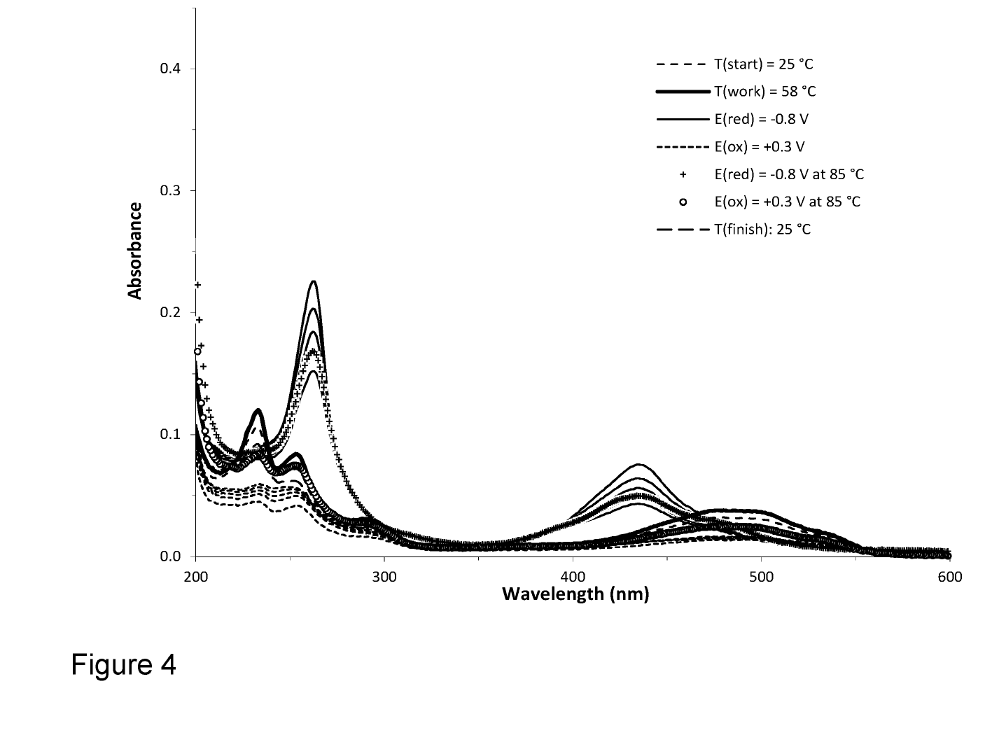

FIG. 4: Electrochemical characterisation of 100 μM DM in 0.036×SSC with spectroelectrochemistry. UV-Vis spectra recorded every 60 s at 58° C. whilst cycling −0.8 V and +0.3 V. λ(DMₒₓ)=233 nm, 253 nm and 480 nm. λ(DM_{red})=262 nm and 435 nm. A constant decrease is observed for reduction peaks.

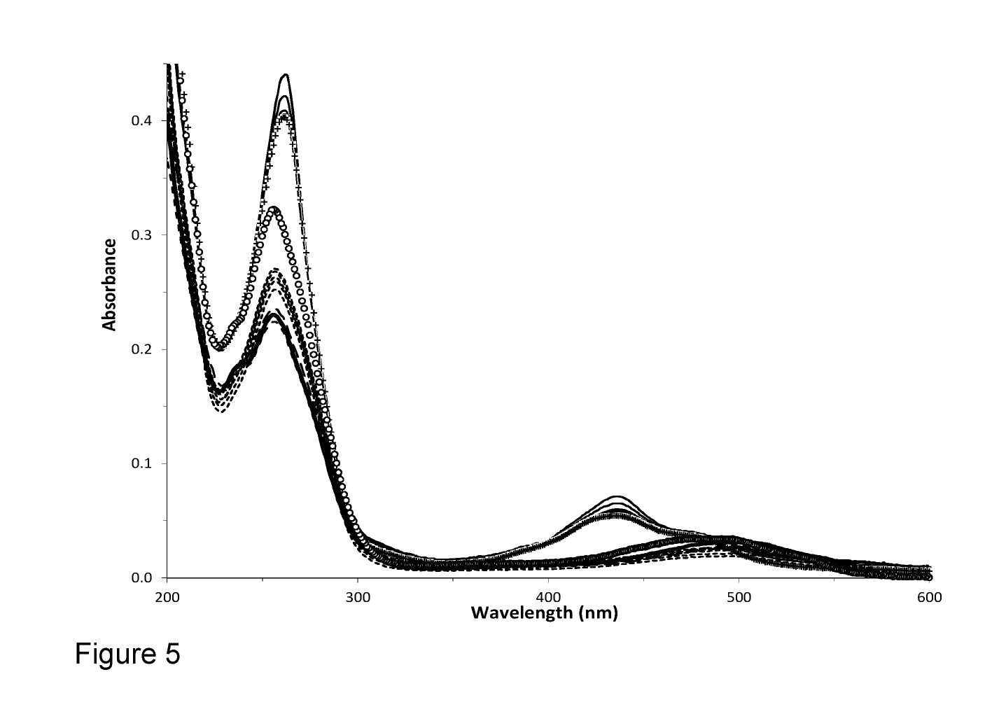

FIG. 5: UV-Vis spectra of 200 μM 20 mer DNA in presence of 100 μM DM (molar ratio 2:1) in 0.036×SSC during cycling of −0.8 V and +0.3 V. Recorded every 60 s at 58° C. Electrochemical DNA denaturation and hybridisation is observed at 260 nm upon cycling the redox-state of DM.

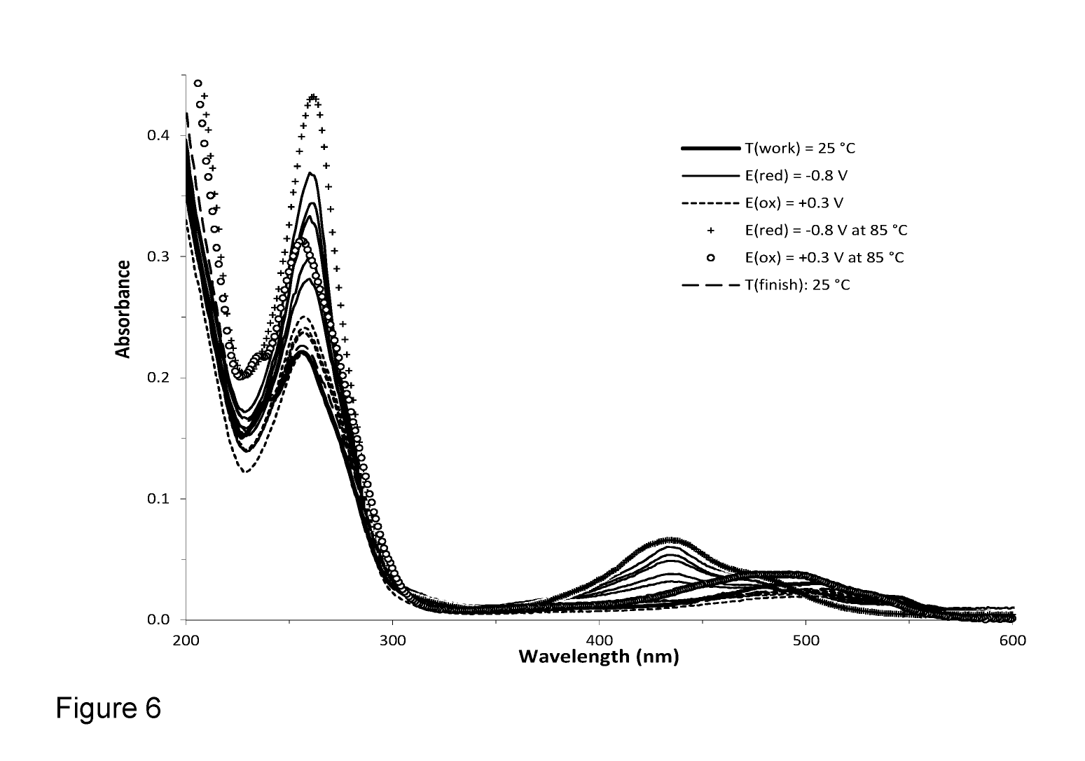

FIG. 6: UV-Vis spectra of 200 μM 20 mer DNA in presence of 100 μM DM (molar ratio 2:1) in 0.036×SSC during cycling of −0.8 V and +0.3 V. Recorded every 60 s at 25° C. (DNA hybridised). Due to considerably smaller changes at 260 nm, DNA denaturation is not observed upon cycling the redox-state of DM.

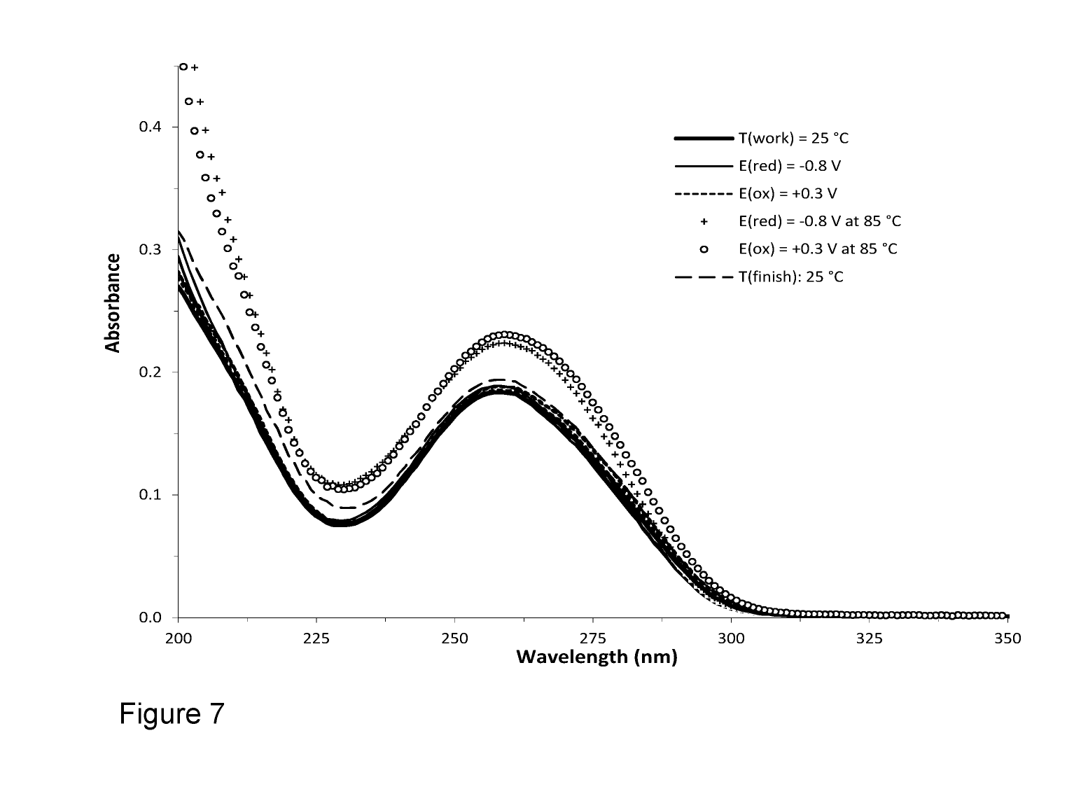

FIG. 7: UV-Vis spectra of 200 μM 20 mer DNA in 0.036×SSC during cycling of −0.8 V and +0.3 V at 25° C. DNA is not affected by potential cycling. Denaturation is only obtained upon increasing to 85° C.

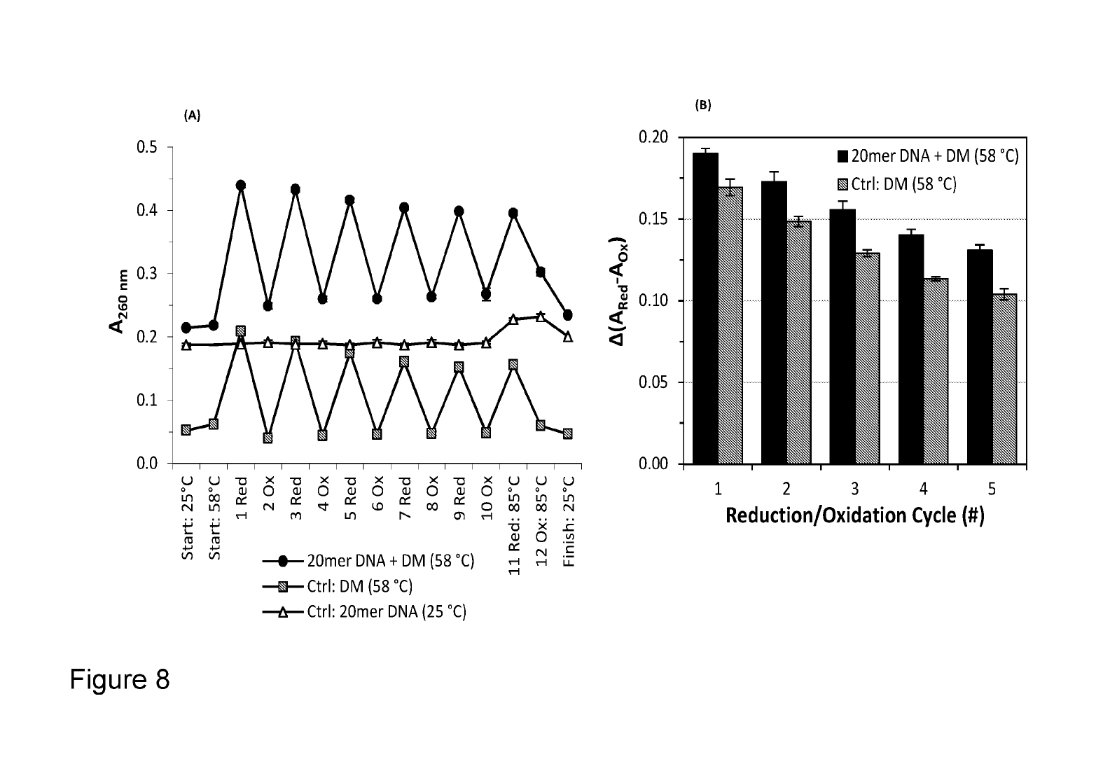

FIG. 8: Switch-diagram of absorbances at 260 nm for 20 mer DNA at 0.036×SSC. (A) Cycling of 20 mer DNA denaturation and hybridisation in presence of DM (n=4), with controls omitting DM (n=3) or DNA (n=3). (B) Comparison of the difference at A_{260 nm} between reduction and oxidation. The increase in absorbance is larger for DNA in presence of DM than for DM alone. Error bars represent standard error.

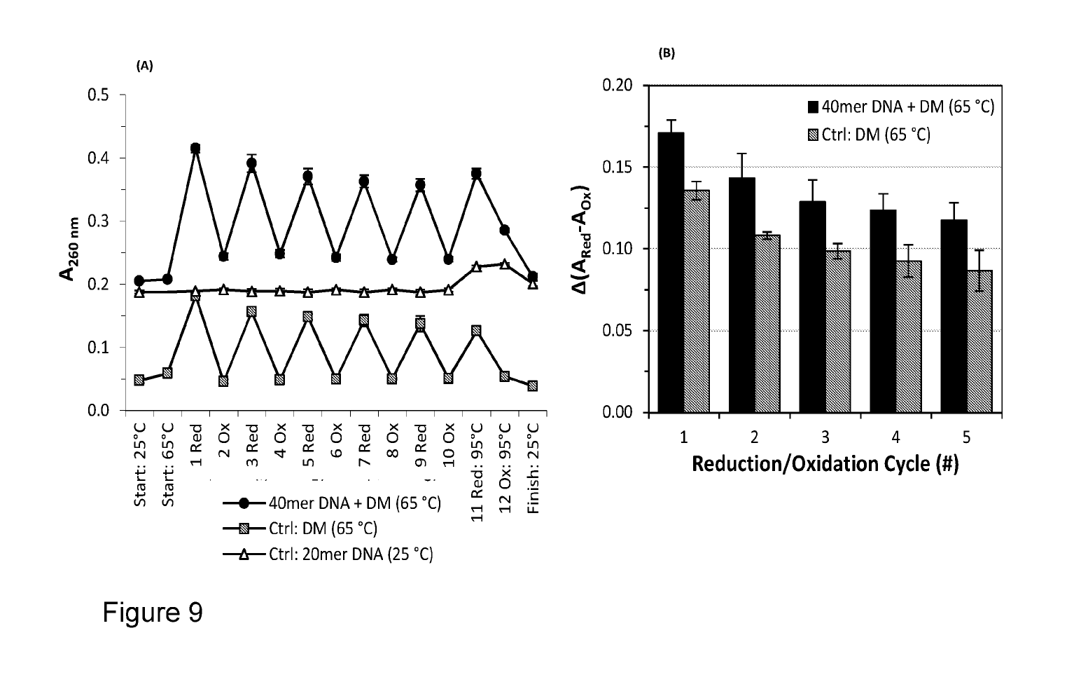

FIG. 9: Switch-diagram of absorbances at 260 nm for 40 mer DNA at 0.036×SSC. (A) Cycling of 40 mer DNA denaturation and hybridisation in presence of DM (n=4), with controls omitting DM (n=3) or DNA (n=3). (B) Comparison of the difference at A_{260 nm} between reduction and oxidation. The increase in absorbance is larger for DNA in presence of DM than for DM alone. Error bars represent standard error.

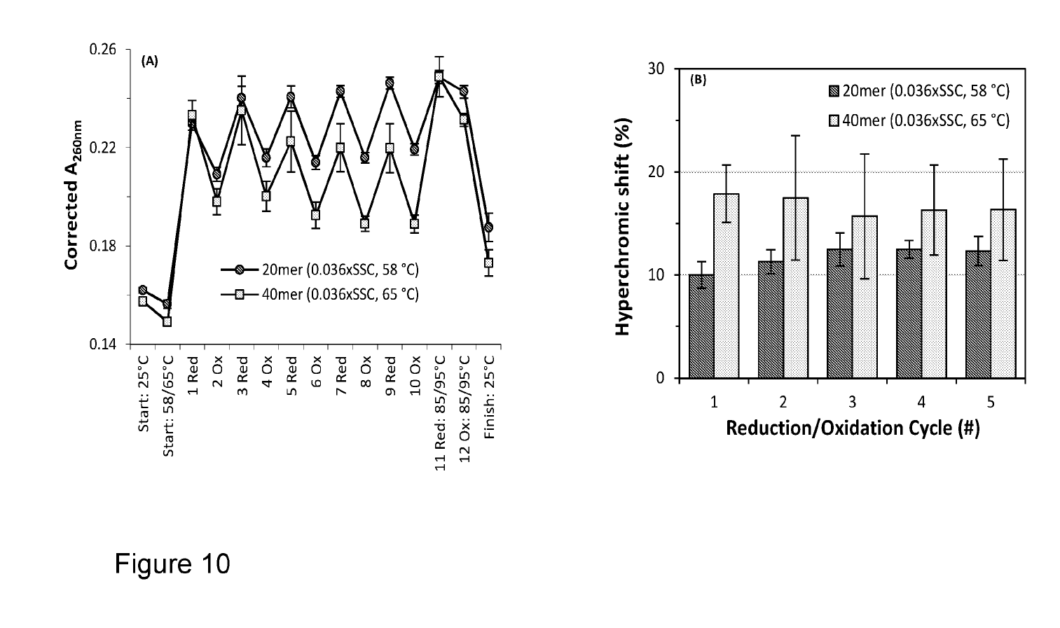

FIG. 10: (A) Summarised absorbances at 260 nm during electrochemical DNA denaturation and hybridisation for 200 μM 20 mer DNA with 100 μM DM in 0.036×SSC at 58° C. (n=4) and 200 μM 40 mer DNA with 100 μM DM in 0.036×SSC at 65° C. (n=3). Data corrected with mean A_{260 nm} obtained from 100 μM DM, in absence of DNA, in 0.036×SSC at 58° C. or 65° C. (n=3). (b) Summarised hyperchromic shifts obtained at each reduction. Error bars represent standard errors.

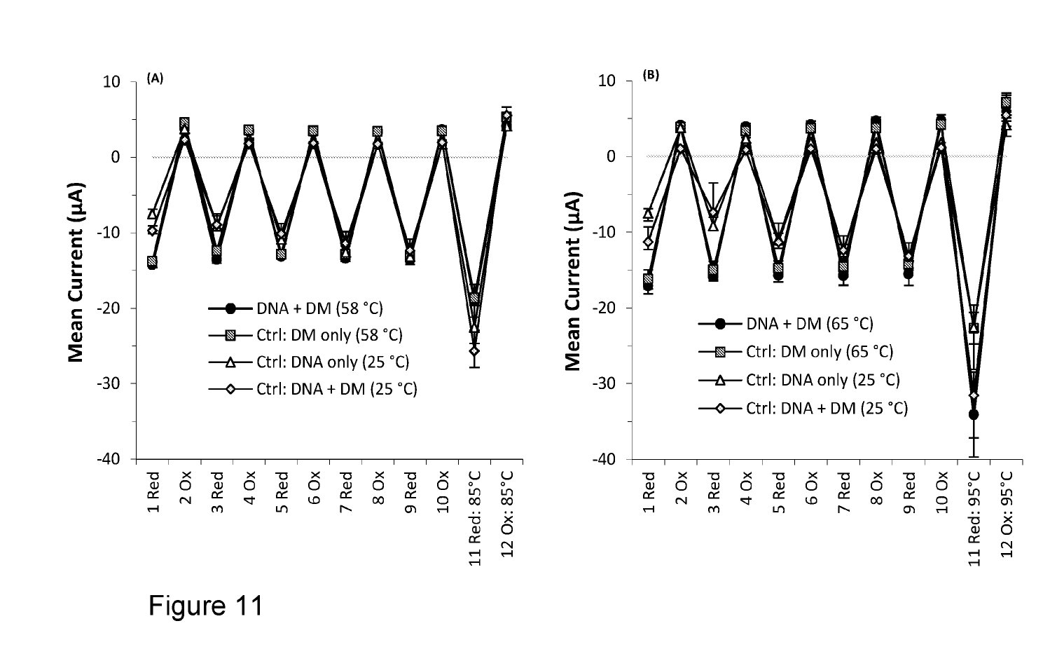

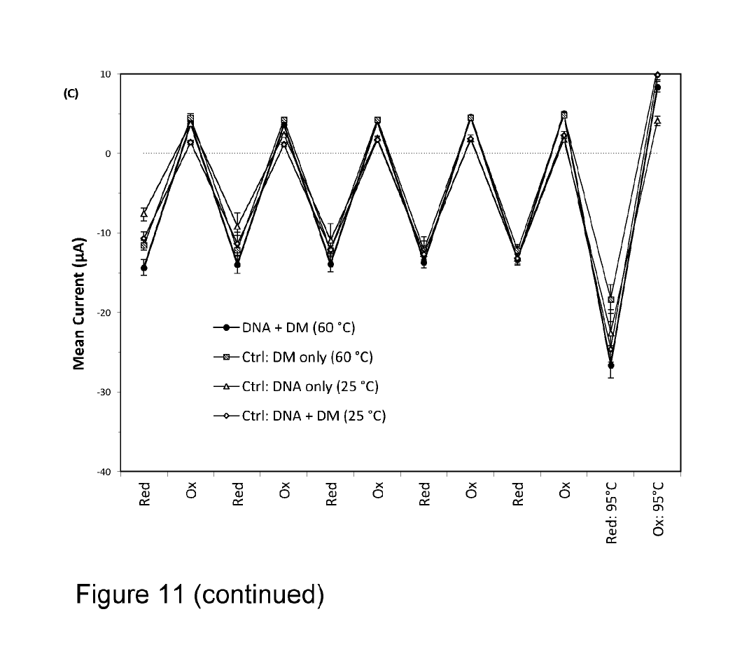

FIG. 11: Reduction and oxidation currents for (A) 20 mer in 0.036×SSC (B) 40 mer in 0.036×SSC and (C) 20 mer in 0.1×SSC during cycling of DNA denaturation and hybridisation. Increasing length of the DNA results in slight increase in currents.

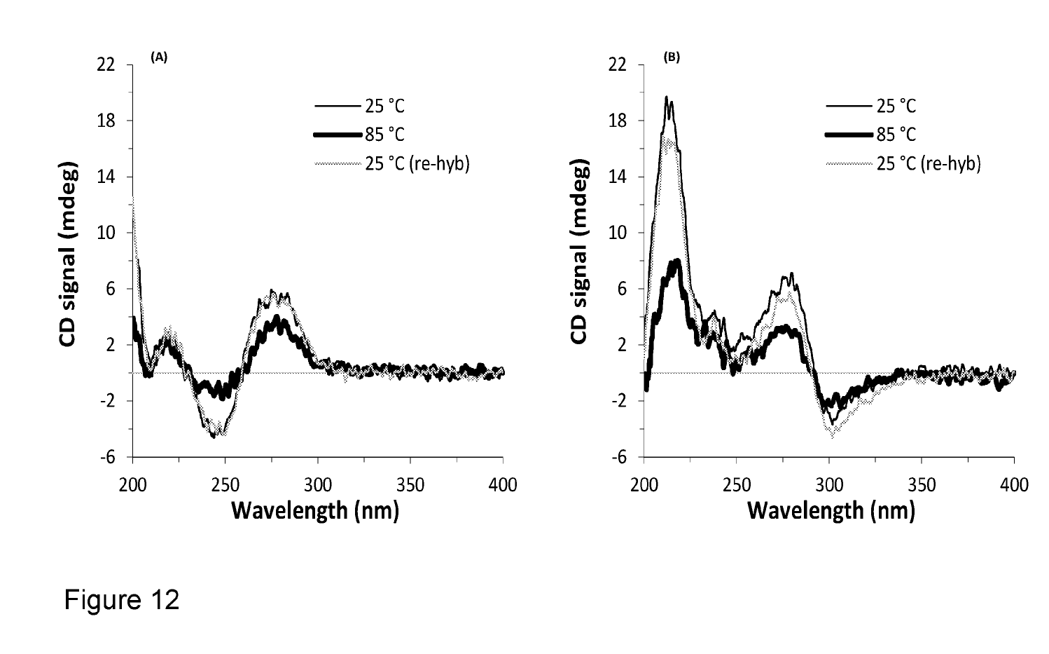

FIG. 12: Circular dichroism (DM) spectra of hybridised DNA at 25° C. and denatured DNA at 85° C. of (A) 200 μM 20 mer DNA in 0.036×SSC and (B) 200 μM 20 mer DNA in presence of 100 μM DM in 0.036×SSC. Change in CD upon denaturation can be seen at 275 nm for both samples.

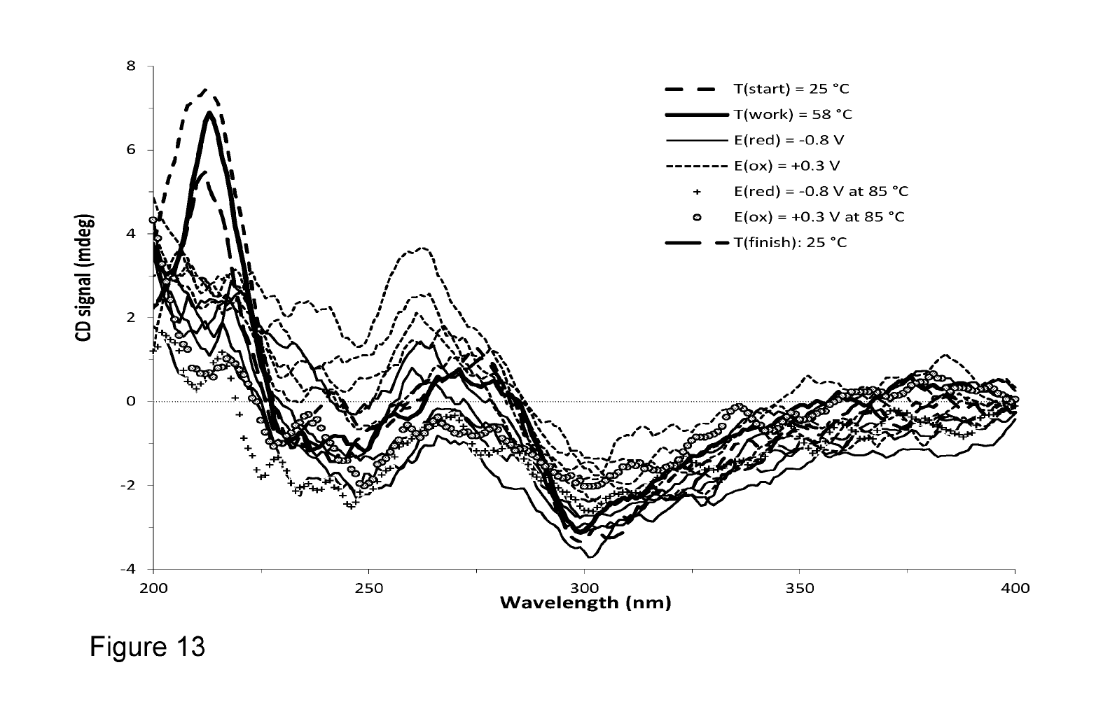

FIG. 13: CD spectra of 20 mer DNA denaturation and hybridisation by cycling the redox-state of DM with −0.8 V and +0.3 V. At 200 μM DNA in presence of 100 μM DM (molar ratio 2:1), in 0.036×SSC, spectra are recorded every 60 s at 58° C. Change is observed at 275 nm. Spectra corrected with the control DM in absence of DNA, normalised to 400 nm and smoothed using the Savitsky-Golay function (convolution width=15).

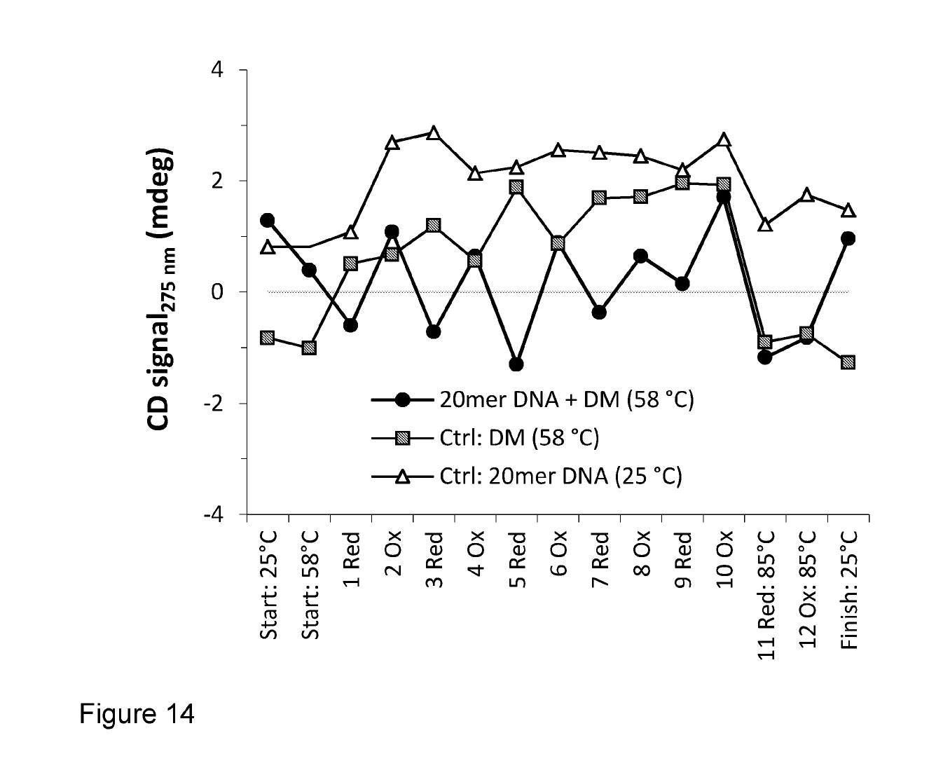

FIG. 14: Switch-diagram of CD signals at 275 nm for 20 mer DNA in presence of DM and two controls where either DNA or DM is omitted. Consistent switching in CD signal, upon cycling −0.8 V and +0.3 V, is only seen for DNA in presence of DM.

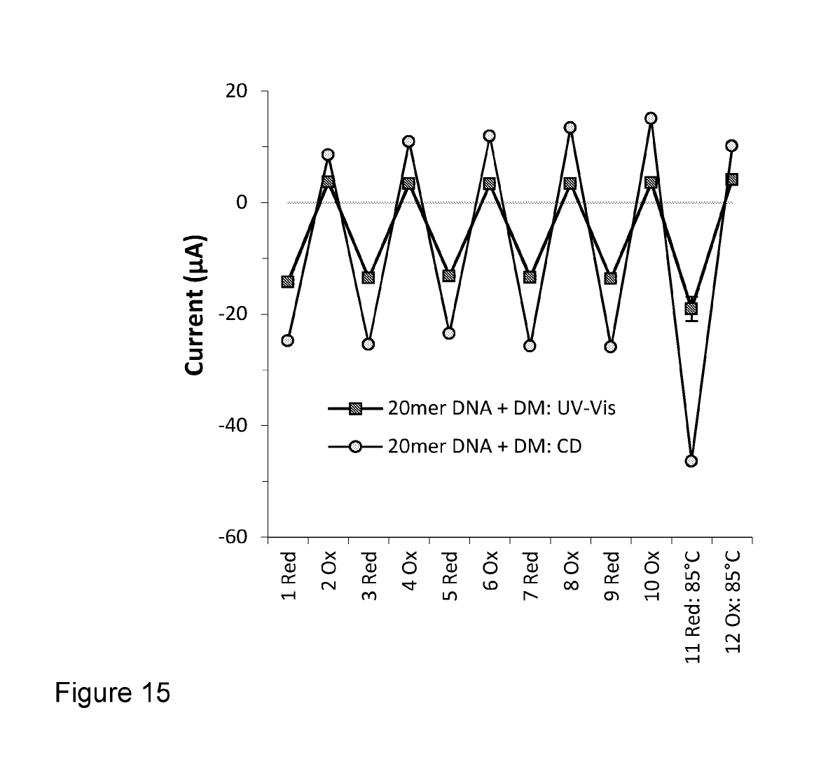

FIG. 15: Reduction and oxidation current during cycling 20 mer DNA denaturation and hybridisation, in presence of DM at 0.036×SSC, performed on UV-Vis or CD.

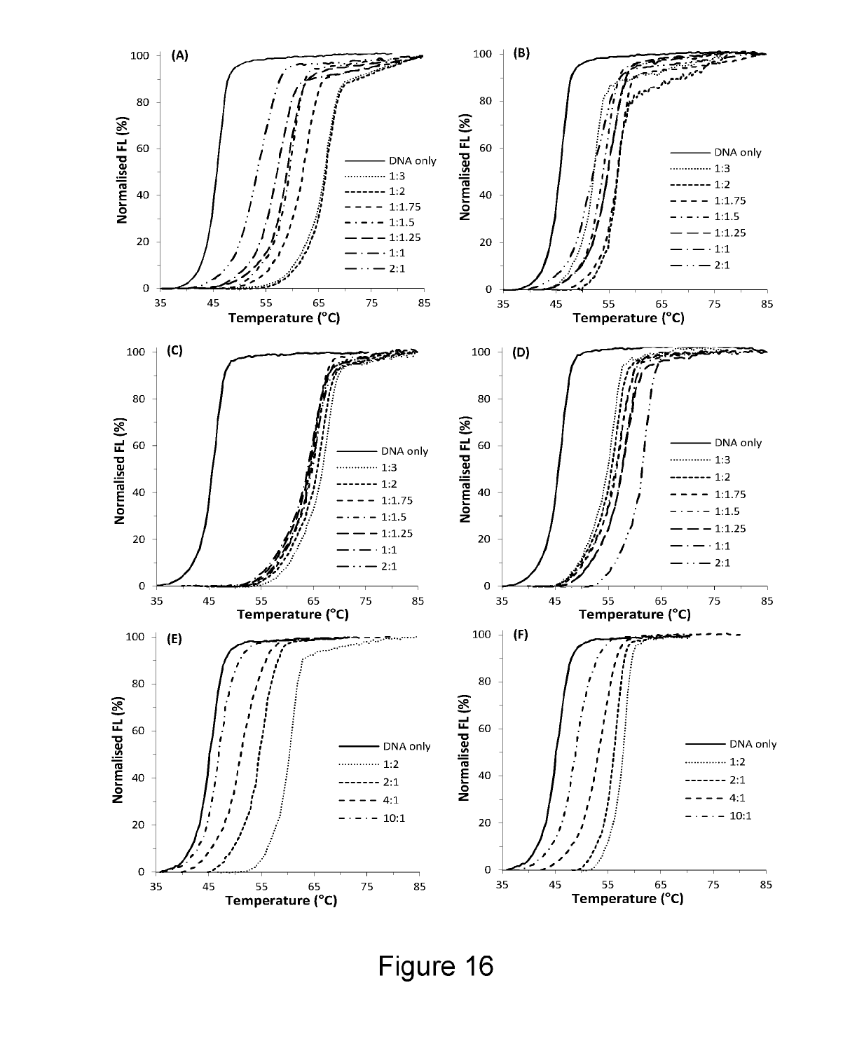

FIG. 16: Fluorescent-based melting curve analysis of 2 μM 20 mer DNA in presence of 6 μM, 4 μM, 3.5 μM, 3 μM, 2.5 μM, 2 μM and 1 μM oxidised/reduced intercalator. DNA in presence of (A) oxidised DM; (B) reduced DM; (C) oxidised AM; (D) reduced AM; (E) oxidised 9-hydroxy ellipticine (9OHE); (F) reduced 9OHE. Molar ratios where oxidised intercalator is in excess show higher T_{M} compared to when intercalator is reduced.

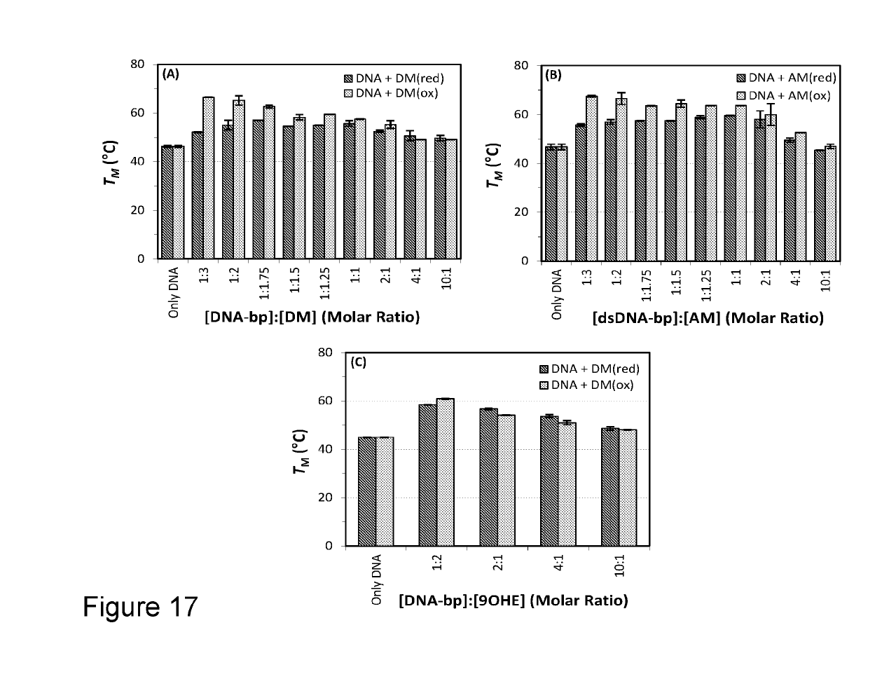

FIG. 17: Comparison of T_{M} for 2 μM DNA in presence 6 μM, 4 μM, 3.5 μM, 3 μM, 2.5 μM, 2 μM, 1 μM, 0.5 μM and 0.2 μM oxidised versus reduced (A) DM; (B) AM and (C) 9OHE.

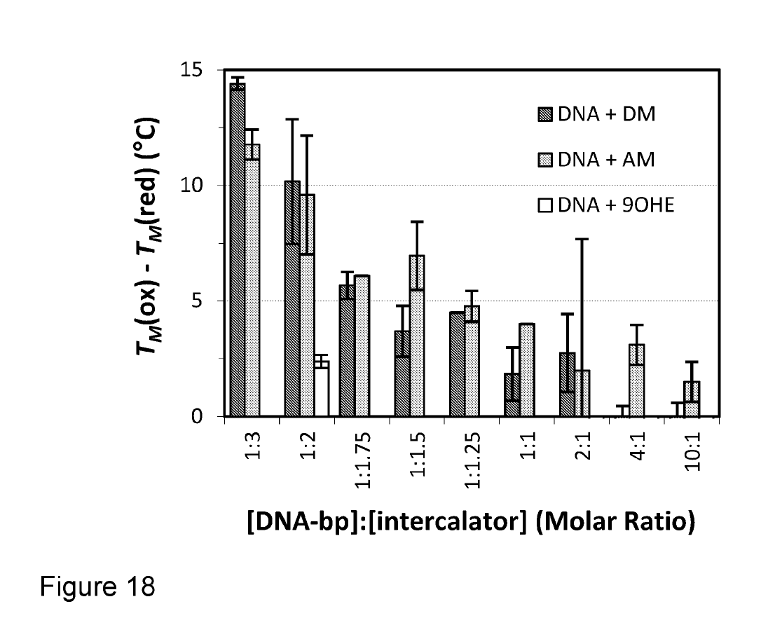

FIG. 18: Difference in T_{M} between DNA with oxidised and reduced DM/AM/9OHE. The largest difference is seen for molar ratio 1:3, i.e. where the intercalator is in excess.

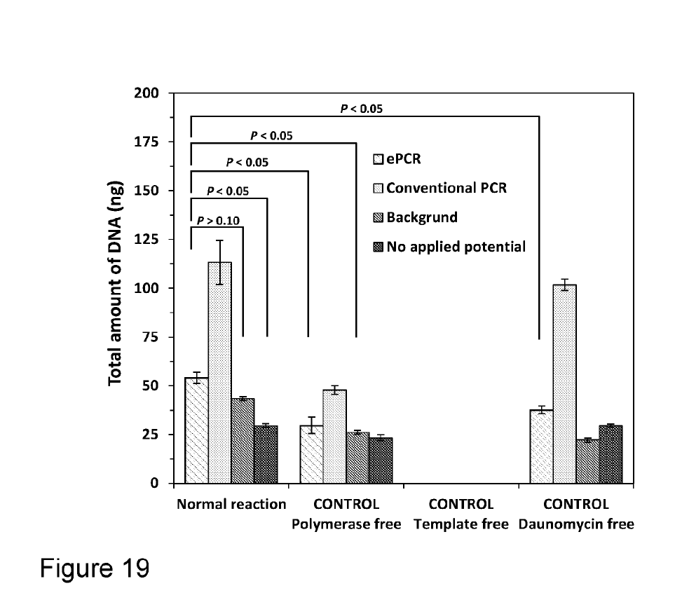

FIG. 19: ePCR-based amplification, with all the relevant controls, on screen-printed carbon electrodes obtained by switching the redox-state of DM, through the application of 20 cycles of the reduction/oxidation potentials −1.3 V/−0.2 V, at the working temperature of 75° C.

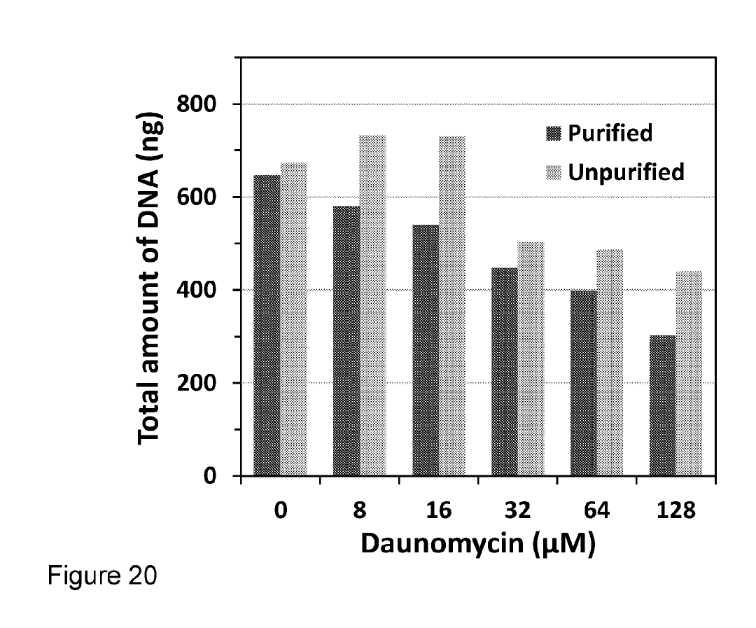

FIG. 20: Gel electrophoretic-based quantification of 40 base pair long dsDNA, in the absence and presence of various DM concentrations, using the Bioanalyzer. Increasing concentrations of DM decreased the ability of the Bioanalyzer to correctly quantify the total amount of DNA.

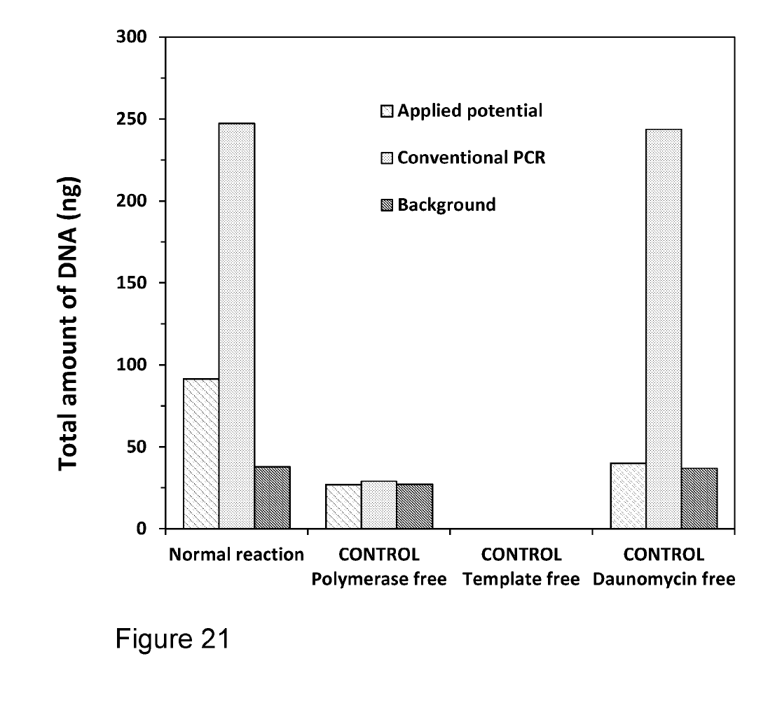

FIG. 21: Optimised ePCR-based amplification, with relevant controls, on screen-printed platinum electrodes.

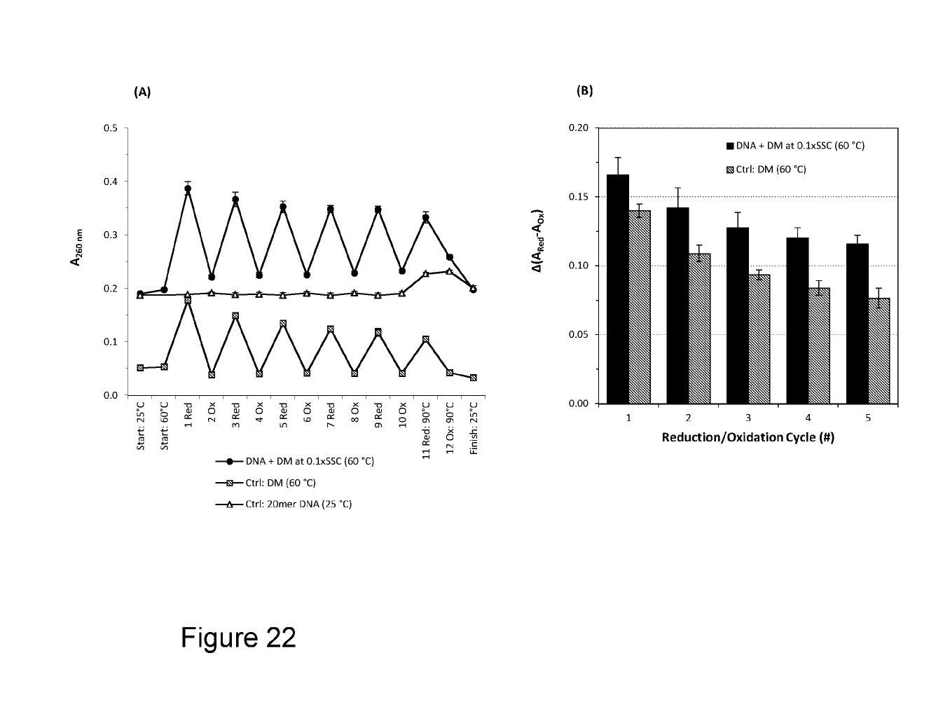

FIG. 22: Switch-diagram of absorbances at 260 nm for 20 mer DNA at 0.1×SSC. (A) Cycling of 20 mer DNA denaturation and hybridisation in presence of DM (n=3), with controls omitting DM (n=3) or DNA (n=3). (B) Comparison of the difference at A_{260 nm} between reduction and oxidation. The increase in absorbance is larger for DNA in presence of DM than for DM alone. Error bars represent standard error.

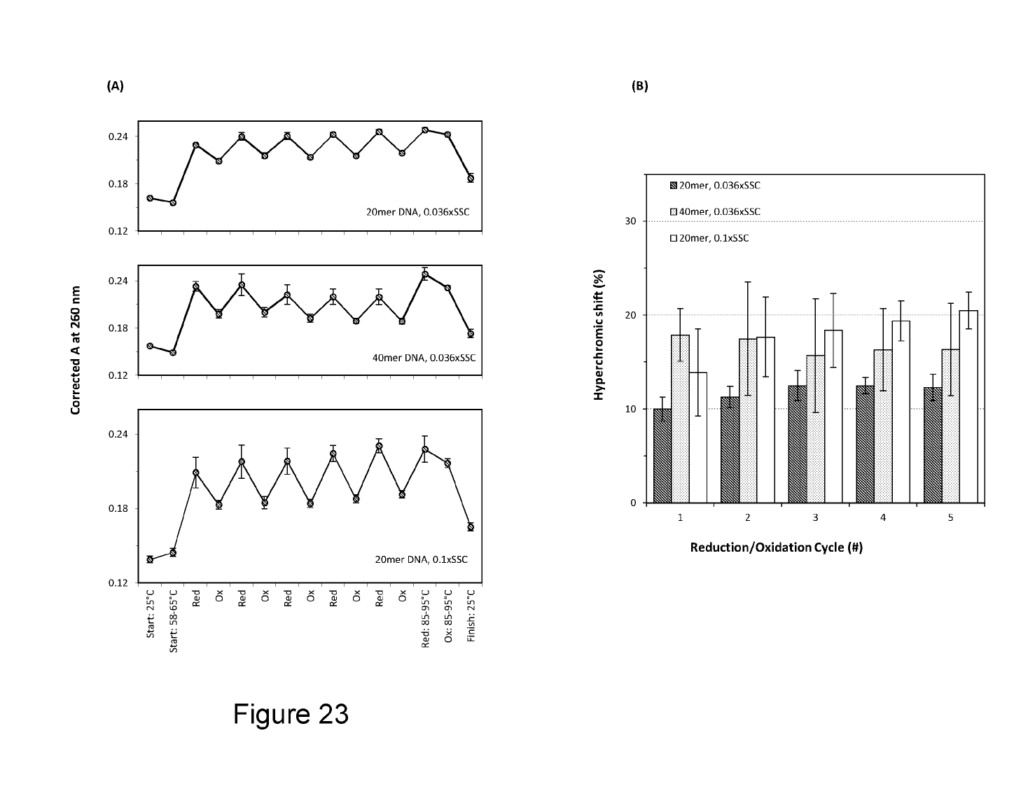

FIG. 23: Summarised absorbances at 260 nm during electrochemical DNA denaturation and hybridisation for 200 μM 20 mer DNA with 100 μM DM in 0.036×SSC at 58° C. (n=4), 200 μM 40 mer DNA with 100 μM DM in 0.036×SSC at 65° C. (n=3) and 200 μM 20 mer DNA with 100 μM DM in 0.1×SSC at 60° C. (n=3). Data corrected with mean A_{260 nm} obtained from 100 μM DM, in absence of DNA, in 0.036×SSC at 58° C., 65° C. or 60° C. (n=3). (b) Summarised hyperchromic shifts obtained at each reduction. Error bars represent standard errors.

### EXAMPLE 1

### Materials and Methods

All solutions were prepared or diluted with ultrapure Milli-Q water (Milli-Q Synthesis, resistance=18.4 MΩ cm, Millipore Corporation, USA). Experiments were conducted in saline-sodium citrate buffer (1×SSC, 0.15 M NaCl with 15 mM trisodium citrate, pH 7 adjusted with HCl, Fisher Scientific, UK) and diluted further as required. Analytical grade ethanol, concentrated nitric acid (HNO₃, VWR Laboratories, UK) and 0.1 M sulfric acid (H₂SO₄, BHD Laboratories, UK) was used for electrode and cuvette cleaning. All electrochemical/spectroelectrochemical experiments were performed with a three-electrode setup in a thin-layer spectroelectrochemical quartz cuvette (pathlength 0.5 mm) (ALS-Japan, Japan). Here, a Pt gauze working electrode (WE) was used with a Pt wire counter electrode (CE) and a single fritted Ag/AgCl (3 M NaCl) reference electrode (RE) (ALS Japan, Japan). Complementary 20 base-pair (bp) long oligonucleotides (sense: 5′-ACA AGG ATG ACA AGC ACA GC-3′, anti-sense: 5′-GCT GTG CTT GTC ATC CTT GT-3′) and 40 bp long oligonucleotides (sense: 5′-GTC GGT CAA GAA CGA GCA CTC AAG AGC CTC AGT CAG ACG A-3′, anti-sense: 5′-TCG TCT GAC TGA GGC TCT TGA GTG CTC GTT CTT GAC CGA C-3′) were purchased from Metabion International AG, Germany. Stock solutions were prepared by dissolving lyophilised DNA in water and stored at −20° C. Concentrations were determined using NanoDrop ND-1000 UV/Vis Spectrophotometer (NanoDrop Technologies Inc., USA). Daunomycin (DM) [(8S,10S)-8-Acetyl-10-[(3-amino-2, 3,6-trideoxy-α-L-lyxo-hexopyransoyl)oxy]-7,8,9,10-tetrahydro-6,8,11-trihydroxy-1-methoxy-5,12-naphthacenedione hydrochloride; λ_{ex/em}=480/592 nm, £_{480 nm}=11 500 M⁻¹ cm⁻¹] was purchased from Tocris Bioscience, UK. Stock solution was prepared by dissolving DM in water, aliquoted, and stored dark at −80° C. The concentration was determined spectrophotometrically. All diluted solutions of DM were freshly prepared for each experiment. All spectrophotometric experiments were conducted on an Agilent Cary 60 UV-Vis spectrophotometer equipped with a Varian Cary Peltier Accessory for temperature control (Agilent Technologies, UK). A Digitron Model 3900 thermocouple (RS Components, UK) was used to monitor the temperature inside the cuvette. Unless stated otherwise, all electrochemical experiments were conducted employing a PalmSens handheld potentiostat/galvanostat (Palmsens, Netherlands). All circular dichroism experiments were performed on a Jasco J-810 Circular Dichroism Spectropolarimeter equipped with a Peltier thermoelectric type temperature controller (Jasco, UK).

### 2.1 Sample Preparation

All experiments were carried out with a 1:1 mixture of complementary oligonucleotides (20 mer or 40 mer) at a by concentration of 200 μM in 0.036×SSC, pH 7 (unless stated otherwise), with or without DM. DNA was hybridised by an initial denaturation at 95° C. for 5 min at 450 rpm followed by cooling down to room temperature over 3.5 h. Samples were stored for maximum a week at +4° C. until used. Intercalation of DM was carried out freshly before each experiment. DM was added to a final concentration of 100 μM resulting in a molar ratio of 2:1. This was incubated at 25° C., 450 rpm, for 10 min protected from light. The solution was degassed for 2.5 min and overlaid for 1 min with argon. The cuvette was cleaned with ethanol and water then finally dried with compressed air. 300 μL was added to the cuvette, followed by electrode assembly (if applied) and overlaying with mineral oil (Biomérieux, UK). To further prevent evaporation the cuvette was sealed with Teflon® tape and Parafilm®.

### 2.2 Cleaning of Electrodes

The Pt gauze WE and Pt wire CE were immersed in concentrated HNO₃ for 5 min at room temperature followed by rinsing with water and drying with a stream of N₂. The WE was cleaned electrochemically in 0.1 M H₂SO₄ using cyclic voltammetry. First, 1.4 V to −0.2 V was cycled 40 times at 50 mV/s followed by 10 times cycling of 1.14 V to −0.24 V at the same speed. Electrodes were finally rinsed with water and dried with a stream of N₂ before use.

### 2.3 DNA Melting Curves

### 2.3.1 UV-Vis Spectrophotometry

Melting curves were obtained for samples containing only DNA, DNA with oxidised DM and DNA with reduced DM and were prepared as in Section 2.1. For samples containing reduced DM, the reduction was obtained chemically using sodium borohydride (NaBH₄, Sigma Aldrich, UK) 50× in excess. Following the hybridisation of DNA and intercalation of DM, reduction took place by adding NaBH₄ to a final concentration of 5 mM. The sample was incubated at 25° C., 450 rpm, for 60 min and then degassed. After using the appropriate buffer as a blank, hybridisation and denaturation was recorded by scanning 200-800 nm at 4800 nm/min (bandwidth 2 nm, interval 1 nm) between 25° C. to 95° C. (depending on sample investigated) at 1° C./min. Absorbance A_{260 nm} was plotted versus temperature T. The melting temperature (T_{M}) was obtained from the peak value of the negative first derivative of the melting curve, i.e. T_{M}=−(dA_{260 nm}/dT)ₘₐₓ. The hyperchromic shift H was calculated according to H_{260 nm}=(A_{Red}−A_{Ox}/A_{Red})×100.

### 2.3.2 Circular Dichroism

Denaturation and hybridisation using circular dichroism was followed by endpoint measurements for samples containing only DNA and DNA with oxidised DM, and were prepared as described in Section 2.1. Here, 400 μL was added to a quartz cuvette with a 1 mm pathlength. CD spectra were recorded at 25° C., 85° C. and then 25° C. again by scanning 200-400 nm at 100 nm/min (data pitch: 0.1 nm, response time: 1 s, bandwidth 1 nm). At the end of the experiments, the appropriate buffer was measured as a blank.

### 2.4 Cyclic Voltammetry

Electroactivity of DM on an Autolab potentiostat (PGSTAT12, Metrohm Autolab, UK). Electrodes were cleaned as described in Section 2.2. DM was diluted to a final concentration of 100 μM in 0.036×SSC and followed by degassing. The potential range of 0 V to −0.8 V was scanned 10 times at 10 mV/s.

2.5 Spectroelectrochemical Switch Experiments with UV-Vis Spectrophotometry

Samples with DNA and DM were prepared as in Section 2.1. The spectrophotometer was blanked using the appropriate buffer, sample added to the cuvette, electrodes assembled and finally the cuvette was sealed. The switch experiment was carried out between 58° C.-65° C. depending on the sample being investigated. A reduction potential (−0.8 V) and an oxidation potential (+0.3 V) was applied for 60 s, respectively, and cycled 5 times each. Spectra were recorded every minute by scanning 800-200 nm at 4800 nm/min (bandwidth 2 nm, interval 1 nm), whereas the current was recorded continuously. The final reduction/oxidation cycle was conducted at 85° C.-95° C. as an internal control. Here, the investigated DNA is expected to be denatured. As a control, the same experiment was carried without DNA, i.e. only 100 μM DM in 0.036×SSC (unless stated otherwise). Additional controls were acquired by conducting experiments with and without DM at 25° C. Here, the investigated DNA is expected to be hybridised throughout the experiment. All samples and controls were measured at least three times.

2.6 Spectroelectrochemical Switch Experiments with Circular Dichroism

Samples with 20 mer DNA and DM were prepared as in Section 2.1. The sample was added to the cuvette, electrodes assembled and finally the cuvette was sealed. The switch experiment was carried out at 58° C. while the reduction potential (−0.8 V) and oxidation potential (+0.3 V) was applied for 60 s, respectively, and cycled 5 times each. CD spectra were recorded, under positive N₂ pressure, every minute by scanning 200-400 nm at 500 nm/min (data pitch 1 nm, response time 0.125 s, bandwidth 2 nm), whereas the current was recorded continuously. As for the switch experiments conducted on the UV-Vis spectrophotometer, see Section 2.5, the final reduction/oxidation cycle was acquired at 85° C. as an internal control. The same experiment was carried out without DNA, i.e. only 100 μM DM in 0.036×SSC (unless stated otherwise) as a control. Additionally, experiments with and without DM at 25° C. were conducted. At the end of the experiments, the appropriate buffer was measured as a blank. All samples and controls were measured at least three times.

### Results

In order to confirm electrochemical control of DNA denaturation and hybridisation using the electroactive intercalator DM, melting curves of DNA with oxidised and reduced form of DM were recorded, while the electroactivity of DM was investigated using cyclic voltammetry. This allowed defining optimal working temperature, as well as oxidation and reduction potentials, for the electrochemical switch experiments employing UV-Vis and CD spectroelectrochemistry. Efficiency of method was investigated by varying length of DNA and salt concentration.

### Melting Curves

Melting curves, recorded spectrophotometrically by monitoring the absorbance at 260 nm (A_{260 nm}), from the three conditions investigated can be seen in FIG. 2 where (A) and (B) represent 20 mer and 40 mer, respectively, in 0.036×SSC (5.94 mM Na⁺) and (C) 20 mer in 0.1×SSC (16.5 mM Na⁺). Melting curves were obtained for DNA alone (control) and DNA in presence of oxidised and chemically reduced DM at 200 μM DNA-bp with 100 μM DM (molar ratio 2:1), regardless of oligonucleotide length. DM was reduced using NaBH₄ in excess before acquiring the melting curve.

Table 1 summarises the T_{M} and hyperchromic shifts of the obtained melting curves. In presence of oxidised DM, the T_{M} of DNA increases significantly. The increase reaches 35° C. for 20 mer in lower salt concentration, while a smaller increase is observed for 20 mer in higher salt concentration. This pronounced increase in T_{M} cannot be observed for DNA in presence of reduced DM. However, at lower salt concentration a slight increase in T_{M} is observed. The difference in T_{M} between DNA in presence of oxidised versus reduced DM for all investigated conditions was found to be almost constant around 25° C. The optimal working temperature to be used in the electrochemical switch experiments was defined as being equal to the temperature where 100% of the DNA, in presence of reduced DM, was in a denatured state. The dashed lines in FIG. 2 represent the optimal working temperatures. Results are shown in

TABLE 1
Table 1. Summary of T_{M} and H values from the DNA melting curves.^{†}

| | DNA | | DNA + DMₒₓ | | DNA + DM_{red} | | | | |
|---|---|---|---|---|---|---|---|---|---|
| | T_{M1} | | T_{M2} | | T_{M3} | | T_{M2} − T_{M1} | T_{M2} − T_{M3} | T_{work} |
| | (° C.) | H (%) | (° C.) | H (%) | (° C.) | H (%) | (° C.) | (° C.) | (° C.) |
| 20mer at | 40.5 | 20.6 | 75.5 | 34.0 | 47.5 | 15.1 | 35 | 28 | 58 |
| 0.036xSSC | | | | | | | | | |
| 40mer at | 51.5 | 24.6 | 83.5 | 34.6 | 58.5 | 14.3 | 32 | 25 | 65 |
| 0.036xSSC | | | | | | | | | |
| 20mer at | 50.5 | 22.6 | 76.5 | 37.4 | 50.5 | 10.1 | 26 | 26 | 60 |
| 0.1xSSC | | | | | | | | | |

^{†}Values obtained from re-hybridisation curves.

### Electrochemical Characterisation of Daunomycin

The electroactivity of 100 μM DM, in absence of DNA was investigated by means of cyclic voltammetry and spectroelectrochemistry. The voltammogram in FIG. 4, obtained at 10 mV/s in room temperature, shows a semi-reversible compound with an oxidation potential E_{c}=−0.41 V and a reduction potential Eₐ=−0.61 V. In order to ensure complete oxidation and reduction of DM in the electrochemical switch experiments, −0.8 V was chosen as reduction potential while +0.3 V was chosen as oxidation potential. The redox-states of DM were then investigated spectrophotometrically using spectroelectrochemistry. FIG. 4 depicts the spectrophotometric changes of DM upon cycling −0.8 V and +0.3 V five times at 58° C. When no potential is being applied (Tₛₜₐᵣₜ, T_{work} and T_{finish}) DM remains oxidised and characteristic peaks at 233 nm, 253 nm and 480 nm can be seen. Upon reduction at −0.8 V, characteristic reduction peaks appear at 262 nm and ˜435 nm. Interestingly, a constant decrease in absorbance is observed at every reduction, whereas at 85° C. it is slightly increased again. Upon re-oxidation at +0.3 V, characteristic oxidation peaks re-appear. However, peaks are less pronounced and slightly shifted at 233 nm, 253 nm and ˜500 nm, which is not the case for re-oxidation at 85° C.

Electrochemical Cycling of DNA Denaturation and Hybridisation Monitored with UV-Vis

FIG. 5 shows the cyclic denaturation and hybridisation of 20 mer DNA in the lower salt concentration (0.036×SSC=5.94 mM Na⁺) obtained with UV-Vis spectroelectrochemistry. The redox-state of 100 μM DM was cycled five times in presence of excess DNA at 200 μM (molar ratio=2:1) at 58° C. Here, spectra were recorded for 25° C. and 58° C., without applying a potential, to ensure that the DNA is not being denatured at the chosen working temperature, followed by cycling −0.8 V and +0.3 V at 58° C. and recording spectra every 60 s. The spectra were recorded within 10 s to minimise UV-radiation time. The temperature was subsequently increased to 85° C. as an internal control, as here, 20 mer DNA is expected to be denatured regardless of applied potential. −0.8 V and +0.3 V was cycled once and spectra were recorded after 60 s. To confirm that the DNA was intact at the end of the cycling, the last scan was performed without applying a potential at 25° C. At no applied potential, two absorbance peaks are visible; at 260 nm associated with DNA and ˜506 nm associated with intercalated DM. Upon applying −0.8 V at 58° C., a constant significant increase in A_{260 nm} for denatured DNA and reduced DM is seen, together with a peak shift to ˜435 nm for reduced DM only. This absorbance increase is reversed upon applying +0.3 V for hybridised DNA and oxidised DM. Furthermore, the peak ˜435 nm shifts back to ˜500 nm for oxidised DM only. As the working temperature is increased to 85° C. and −0.8 V applied, DM reduces and the 20 mer DNA is expected to be denatured. As can be seen, A_{260 nm} at 85° C. overlaps with A_{260 nm} obtained at 58° C. Switching to +0.3 V, DM re-oxidises. As mentioned earlier, DNA is expected to stay denatured and thus A_{260 nm} is higher at 85° C. than at 58° C. Moreover, the peak for oxidised DM has increased in absorbance and shifted back to 480 nm for free DM.

In contrast, conducting the entire experiment at 25° C., the 20 mer DNA is expected to be hybridised regardless of applied potential. shows the observed increase in A_{260 nm}, upon applying −0.8 V, which is considerably smaller compared to 58° C.

FIG. 6 shows that the investigated potentials of −0.8 V and +0.3 V do not affect and cause any changes to the 20 mer DNA in absence of DM. Denaturation is only observed as the temperature is increased to 85° C., regardless of applied potential.

The values at A_{260 nm} for each reduction and oxidation from FIGS. 5, 6 and 7 are summarised in a switch-diagram in FIG. 8(A). As can be seen, the increase in A_{260 nm} upon reduction of DM in absence of DNA is significant, while none is observed for DNA alone. FIG. 8(B) elucidates the difference in the switching of A_{260 nm} between DNA in presence of DM and DM in absence of DNA. A clear difference is observed at every reduction/oxidation cycle suggesting it arising from the denaturation of DNA.

The efficiency of the method was studied by increasing the length of the DNA. An identical experimental setup was employed to investigate 40 mer DNA at 0.036×SSC, however, the experiment was conducted at 65° C. (see FIG. 2. (B)). FIG. 9(A) summarises A_{260 nm} obtained from DNA in presence DM and DM in absence of DNA, whereas FIG. 9(B) reveals the difference observed in the switching. As seen for the 20 mer DNA, a consistent difference in absorbance is observed at every reduction/oxidation cycle for the 40 mer DNA, hence, correspondingly suggesting it arising from the denaturation of DNA. Moreover, the efficiency of the method was also studied for the 20 mer in an increased salt concentration (0.1×SCC (16.5 Na⁺)). FIG. 22 shows the difference observed in switching at high salt concentration (0.1×SCC), performed at 60° C.

While the signal at A_{260 nm} obtained for 20 mer or 40 mer DNA (at 0.036×SSC=5.94 mM Na⁺). in presence of DM in FIG. 8 and FIG. 9, respectively, shows the combined absorbance of the two types of molecules. Similarly, the signal at A_{260 nm} obtained for 20 mer at 0.1×SCC (16.5 Na⁺) in presence of DM is shown in FIG. 22. FIG. 10(A) illustrates the observed A_{260 nm} values corrected with the mean A_{260 nm} (n=3) observed for DM in absence of DNA, at given conditions. In FIG. 10(A) it is clearly observed that A_{260 nm} is increased upon reduction and decreased upon oxidation revealing DNA denaturation and hybridisation of both the 20 mer and 40 mer DNA in presence of DM (molar ratio 2:1), at lower salt concentration (0.036×SSC=5.94 mM Na⁺). The hyperchromic shift for each reduction/oxidation cycle, represented in FIG. 10(B), averages at ˜12% for the 20 mer DNA. Although the reproducibility has suffered slightly, ˜17% is obtained for the 40 mer DNA. The hyperchromic shift, obtained with thermal denaturation/hybridisation (Table 1), is 20.6% for the 20 mer DNA and 24.6% for the 40 mer DNA, which corresponds to ˜55% and ˜67%, respectively, of the DNA being electrochemically denatured and hybridised at given conditions. Similarly, FIG. 23(A) shows that A_{260 nm} is increased upon reduction and decreased upon oxidation revealing DNA denaturation and hybridisation of the 20 mer DNA in presence of DM (molar ratio 2:1), at higher salt concentration (0.1×SCC (16.5 Na⁺)). FIG. 23(B) shows an average ˜18% increase in the hyperchromic shift of the 20 mer at higher salt concentration 0.1×SCC (16.5 Na⁺), which corresponds to ˜80% of the DNA being electrochemically denatured and hybridised at the given conditions.

The recorded currents during cycling DNA denaturation and hybridisation are shown in FIG. 11 for (A) 20 mer; (B) 40 mer in 0.036×SSC and (C) 20 mer in 0.1×SCC. For the three experimental conditions, it is evident that the oxidation current is smaller than the reduction current. Furthermore, upon increasing the temperature, an increase in the current is observed, whereas only slight differences can be seen between the two different lengths of DNA.

Electrochemical Cycling of DNA Denaturation and Hybridisation Monitored with Circular Dichroism (CD)

To corroborate results obtained with UV-Vis spectroelectrochemistry, experiments were performed utilising CD spectroelectrochemistry to record DNA denaturation and hybridisation upon cycling the redox-state of DM electrochemically.

Before switch experiments were conducted, the change in CD upon DNA denaturation and hybridisation of 20 mer DNA was investigated by endpoint-measurements at 25° C. and 85° C. FIG. 12(A) shows the CD spectra from 20 mer DNA in absence of DM. At 25° C., when hybridised, the DNA displays strong positive CD signals at 220 nm and ˜275 nm, while a strong negative CD signal is obtained ˜250 nm. Upon denaturation, at 85° C., CD signals at 275 nm and 250 nm decrease. FIG. 12(B) shows the CD spectra from 20 mer DNA in presence of oxidised DM at a molar ratio 2:1. When hybridised, at 25° C., strong positive CD signals at 215 nm and ˜275 nm are found. A dip is observed ˜250 nm while a strong negative CD signal is seen at 300 nm. Upon denaturation, at 85° C., CD signals at 215 nm, ˜275 nm and 300 nm decrease. Due to the decrease in the CD signal at ˜275 nm upon denaturation, for DNA in absence and presence of oxidised DM, this signal is monitored in the switch experiments.

FIG. 13 shows the cyclic denaturation and hybridisation of 20 mer DNA in 0.036×SSC (i.e. 5.94 mM Na⁺) obtained with CD spectroelectrochemistry. Identical experimental condition and workflow was employed here as for cyclic denaturation and hybridisation of 20 mer DNA investigated with UV-Vis spectroelectrochemistry (FIG. 5). The redox-state of DM was cycled five times at 58° C. Upon cycling −0.8 V and +0.3 V, a consistent change at 275 nm and 300 nm is observed. As the working temperature was increased to 85° C. and the redox-potentials applied, the obtained spectra overlap with the ones obtained at 58° C. during DM reduction. The observed change at 275 nm is represented in a switch-diagram in FIG. 14 along with two controls. A clear switching of the CD signal, upon cycling the reduction and oxidation potential, can be observed for the sample containing 20 mer DNA in presence of DM. A similar consistent switching is not observed in samples omitting either DM or DNA, thus suggesting that the observed switching at 275 nm for DNA in presence of DM arises from DNA denaturation and hybridisation. FIG. 15 compares the recorded current between the switch experiment performed with CD spectroelectrochemistry and UV-Vis spectroelectrochemistry where the former shows higher currents.

FIG. 16 shows a screening of the effect of various oxidised and chemically reduced (with NaBH₄) intercalators on the stability of DNA. Fluorescence-based melting curve analysis of 20 mer DNA was conducted in the presence of (A) oxidised DM and (B) reduced DM; (C) oxidised AM and (D) reduced DM; (E) oxidised 9OHE and (F) reduced 9OHE. An increasing melting temperature can be observed for DNA in the presence of an oxidised form of intercalator, while a similar increase is not observed in the presence of reduced intercalator. The summarised effect on the melting temperature of DNA, of the various intercalators, can be seen in FIG. 17 for (A) DM, (B) AM and (C) 9OHE. As observed, a larger difference in the melting temperature, between DNA in the presence of oxidised versus reduced intercalators, is seen with increasing concentrations of the intercalator. A clearer visualisation of the difference can be observed in FIG. 18. It is evident that DM yields the largest difference. However, an almost equal difference is observed for AM, while 9OHE yields the lowest difference.

### Discussion and Conclusion

Electrochemically cycled DNA denaturation and hybridisation by cycling the redox-state of DM was first investigated using spectroelectrochemistry. Here, UV-Vis spectra was recorded while applying a reduction or oxidation potential (−0.8 V/+0.3 V). The strength in this technique lies in that the behaviour of DNA and DM can be followed. Denaturation and hybridisation of DNA was followed at 260 nm, while DM intercalation was followed between 400-500 nm. As the reduction potential was applied, FIG. 5 shows an increase at 260 nm and the appearance of a peak at ˜435 nm. The first peak is the combined absorbance from denatured DNA and reduced DM while the second peak is the absorbance from reduced DM only. As the oxidation potential was applied, the peak at 260 nm decreased and the peak at ˜435 nm shifted back to ˜500 nm. Likewise, the first peak is the combined absorbance from hybridised DNA and oxidised DM while the second peak is the absorbance from intercalated oxidised DM. Denaturation and hybridisation of DNA was concluded from FIG. 8(B), where it is evident that the change in absorbance upon switching the redox-state of DM alone is smaller than the switching obtained of DM in presence of DNA. That is, the extra absorbance obtained when DM is in presence of DNA must be from denatured DNA, since denatured DNA absorb more than hybridised DNA. The state of the DNA was equally determined by increasing the temperature of the experiment to 85° C. As stated before, at this temperature the DNA is expected to be denatured regardless of the redox-state of DNA, see the melting curves in FIG. 2. As the reduction potential was applied at 85° C., see FIG. 5, the peak at 260 nm for DNA and reduced DM reached a similar absorbance as when the reduction potential was applied at the working temperature (i.e. 58° C., 60° C. for 20 mer at 0.036×SCC or 0.1×SCC respectively or 65° C. for the 40 mer DNA at 0.036×SCC). Additionally, as the oxidation potential was applied at 85° C., the peak for oxidised DM appeared with an increased absorbance at 480 nm. In other words, since the DNA is no longer hybridised, DM can no longer intercalate. Thus, indicating that oxidised DM was indeed intercalated into hybridised DNA at the working temperatures (i.e. 58, 60 or 65° C.), since here the peak for oxidised DM appeared with less absorbance at ˜500 nm. Similar behaviour was observed for experiments with 20 mer and 40 mer DNA at lower salt concentration (0.036×SCC (5.9 mM Na⁺)), see FIG. 10. However, the variability for 40 mer DNA is larger than for 20 mer DNA. It can be seen that the absorbance values obtained for denatured DNA in presence of reduced DM are not as high for 40 mer DNA as for 20 mer DNA, whereas the absorbance values obtained for hybridised DNA in presence of oxidised DM are not as low for 20 mer DNA as for 40 mer DNA. For the 20 mer at a higher salt concentration (0.1×SCC (16.5 mM Na⁺)) there is low variability. The absorbance values in presence of reduced DM are not as high as those obtained for the 20 mer at low salt concentration but the absorbance values in the presence of oxidised DM are lower as those of the 20 mer at low salt concentration. Since all three samples contain the same concentration of DNA bp, the values should theoretically overlap. Again, without wishing to be bound by theory, this difference may arise as it may be easier to hybridise longer DNA and/or DNA at a higher salt concentration, i.e. the strands are more likely to find each other hence resulting in lower absorbance values for the 40 mer DNA and at a higher salt concentrations dsDNA is more stabilised. Equally, it may be easier to denature short DNA since there are fewer base pairs that can contribute to stabilising forces in the double helix, hence resulting in higher absorbance values for 20 mer DNA.

The denaturation and hybridisation was furthermore confirmed with CD spectroelectrochemistry. FIG. 13 shows a clear difference in the spectra obtained for hybridised DNA upon the application of oxidation potential versus denatured DNA upon the application of reduction potential. However, due to the nature of the experiment where a fast denaturation and hybridisation is required, the obtained CD spectra are very noisy. The CD spectra were collected at a high scan speed and low response time which sacrificed the resolution. Therefore FIG. 14, which summarises the switch experiment of the 20 mer DNA with the relevant controls, shows very noisy data. Nevertheless, it can clearly be seen that a constant switching is only obtained for the sample with DNA in presence of DM. To verify these results and obtain significance, the experiment needs to be repeated.

While denaturation and hybridisation was confirmed with DM, FIG. 17 suggests that other compounds which behave similarly to DM may be equally utilised for the electrochemical control of DNA denaturation and hybridisation. These may be AM and 9OHE.

It can be concluded that both DNA denaturation and hybridisation can be electrochemically controlled by cycling the redox-state of DM, and thereby circumventing the need for cycling temperature. This was first confirmed using a short 20 mer DNA strand at a low salt concentration, where ˜55% of the DNA in solution was being denatured and hybridised in a cyclic mode. To investigate to effect of the length of the DNA strand and the salt concentration, identical experiments were performed with a 40 mer DNA strand at low salt concentration and a 20 mer DNA strand at a higher salt concentration. Here, ˜67% and ˜80% respectively, of the DNA in solution was being electrochemically denatured and hybridised. The redox-state of DM was switched five times, corresponding to five cycles of DNA denaturation and hybridisation obtained at 144 s/cycle was obtained in under 12 minutes, rendering this the application of this technique highly advantageous portable nucleic acid-based diagnostics, exploiting established nucleic acid amplification techniques.

### EXAMPLE 2

### (Un-Optimised) Electrochemical PCR (ePCR)

### Materials and Methods

A total ePCR reaction mixture of 175 μL included the following components:
0.2 μM forward primer
0.2 μM reverse primer
200 μM of each dNTP
10 mM Sodium-Phosphate Buffer (pH 8.6)
1.2 mM MgCl₂
0.04 μM or 0.49 ng/μL of double stranded NDM template (40 bp)
8 μM DM
1.25 U Dynazyme I DNA Polymerase (Thermo Scientfic)

Prior to performing ePCR, the screen-printed carbon electrode was activated in a mixture of 100 mM KCl and 50 mM PBS by applying +1.7 V for 3 min. A hot plate had been calibrated to the working temperature of 75° C. A reaction well was next attached to the electrode, into which the ePCR sample mixture was transferred. The well was properly sealed using a sticky aluminium lid, connected to a box connector and then placed on top of the hot plate. After allowing the sample mixture to equilibrate to the working temperature for 5 min, 20 consecutive cycles of the oxidation potential Eₒₓ=−0.2 V and reduction potential E_{red}=−1.3 V were applied. The first cycle consisted of applying Eₒₓ and E_{red} for 120 s each. Subsequent cycles consisted of applying Eₒₓ and E_{red} for 60 s each. The total experimental time was 45 min. After the applied potential cycles, the sample was recovered and DNA amplicons were purified using the GeneJET PCR Purification Kit. Purified samples were then analysed using capillary gel electrophoresis. The amplicons were sized in number of base pairs and quantified in total amount of DNA.

Each ePCR investigation included four sets of samples, which are explained in the list below:
1. ePCR: the ePCR reaction mixture was prepared as above and amplification was conducted according to the measurement sequence explained above
2. Conventional PCR (control): the ePCR reaction mixtures prepared as above and used for amplification in a conventional thermocycler using the standard sequence of three different temperatures to drive the reaction
3. Background (control): the ePCR reaction mixture was prepared as above and immediately purified without undergoing any amplification reaction/measurement sequence
4. No applied potential (control): the ePCR reaction mixture and electrodes were prepared as above, however, no reduction/oxidation potentials were applied, the mixture was incubated at the working temperature for 45 min, which is equivalent to application of 20 cycles of the reduction/oxidation potential

For each set of sample (1-4) in the list above, the sample reaction (A) was concurrently analysed with its own series of controls (B-D) as listed below:

A. Normal reaction: includes template, primers, polymerase, DM

B. Polymerase free (control): includes template, primers, DM→No polymerase

C. Template free (control): includes primers, polymerase, DM→No template

D. Daunomycin free (control): includes template, primers, polymerase→No DM

All experiments were performed on an Autolab potentiostat from Metrohm (Runcorn, UK). All screen-printed electrodes were purchased from DropSens (Spain) and consisted of a three-electrode setup with a carbon or platinum working electrode, platinum counter electrode and AgCl reference electrode. Purification of ePCR samples were conducted using the GeneJET PCR Purification Kit (Thermo Scientific, Massachusetts, USA). Gel electrophoretic analysis was performed on the Agilent 2100 Bioanalyzer (Agilent Technologies, Santa Clara, USA).

### Results

### Confirmation of Amplification

FIG. 19 shows the results for the ePCR performed on screen-printed carbon electrodes. A statistically significant difference is observed between the ePCR ‘normal reaction’ and the control lacking the polymerase and DM, thus, indicating that amplification of the 40 base pair long NMD target was amplified. ePCR-based amplification was obtained by switching the redox-state of DM, through the application of 20 cycles of the reduction/oxidation potentials −1.3 V/−0.2 V, at the working temperature of 75° C. Error bars represent standard error (n=3). P-values are calculated based on the two-tailed Student's t-test (95% confidence interval), where P<0.05 shows a statistical difference.

The ePCR ‘normal reaction’ which contained all components, contained more DNA than the control lacking the polymerase and DM. To test if the amplification in the ‘normal reaction’ was significantly higher from the ‘polymerase free’ and ‘DM free’ control, the two-tailed Student's t-test was performed where a P-value below 0.05 showed that the tested samples were statistically significant different. This was indeed confirmed upon obtaining P<0.05. Thus, evidence indicated that amplification had taken place in the ‘normal reaction’ and could specially be attributed to the presence of DM. Furthermore, a significantly higher degree of amplification (P<0.05) was observed in the ePCR ‘normal reaction’ than in the control set where no potential was applied. This implied that the amplification observed in the ePCR ‘normal reaction’ only took place due to the redox-state switching of DM which electrochemically controlled the denaturation and hybridisation of DNA. The observed ePCR-based amplification in the ‘normal reaction’ was further substantiated by obtaining a significantly lower degree of amplification (P<0.05) in the background control samples. Although, it should be noted that no significant difference was observed when the ePCR ‘normal reaction’ was compared to the background ‘normal reaction’, thus, suggesting variability in the background control samples. Finally, considering that the ePCR method has not been fully optimised, the ePCR amplified DNA reached acceptable levels when compared to the benchmark which is conventional PCR.

### Nucleic Acid Detection

FIG. 20 shows the gel electrophoretic-based quantification of a 40 bp long dsDNA, in the absence and presence of various DM concentrations, using the Bioanalyzer. It was noted that increasing concentrations of DM decreased the ability of the Bioanalyzer to correctly quantify the total amount of DNA. Specifically, the total amount of DNA detected in the absence of DM was 600 ng. Upon increasing amounts of DM, the total amount of detected DNA decreased. This confirmed that DM, which is an intercalator, interfered with the intercalator based detection method used in the Bioanalyzer. Without wishing to be bound by theory it is suggested that the interference may result from quenching of fluorescence or through residual DM intercalation.

### EXAMPLE 3

### Modified Electrochemical PCR (ePCR)

### Material and Methods

The ePCR method was optimised by including an additional step of surface-coating of the electrodes with BSA prior to the ePCR experiment. In this procedure, screen-printed platinum electrodes from DropSens (Spain) were used and the concentration of DNA in all samples and controls was higher than in the non-optimised method of Example 2. The electrodes were not cleaned prior to the measurements. A hot plate had been calibrated to a working temperature of 75° C. A reaction well was next attached to the electrode, into which the ePCR sample mixture was transferred. The well was properly sealed using a sticky aluminium lid, connected to a box connector and then placed on top of the hot plate. After attaching the well to the electrode, 133 μL of 0.5 μg/μL BSA for 5 min was added in order to coat the surfaces. The solution was removed with a pipette prior to adding the ePCR mixture. After allowing the sample mixture to equilibrate to the working temperature for 5 min, 20 consecutive cycles of the oxidation potential Eₒₓ=−0.2 V and reduction potential E_{red}=−1.3 V were applied. The first cycle consisted of applying Eₒₓ and E_{red} for 120 s each. Subsequent cycles consisted of applying Eₒₓ and E_{red} for 60 s each. The total experimental time was 45 min. After the applied potential cycles, the sample was recovered and DNA amplicons were purified using the GeneJET PCR Purification Kit. Purified samples were then analysed using capillary gel electrophoresis. The amplicons were sized in number of base pairs and quantified in total amount of DNA.

A total ePCR reaction mixture of 175 μL included the following components:
0.2 μM forward primer
0.2 μM reverse primer
200 μM of each dNTP
10 mM Sodium-Phosphate Buffer (pH 8.6)
1.2 mM MgCl₂
0.04 μM or 0.49 ng/μL of double stranded NDM template (40 bp)
8 μM DM
2 U Dynazyme I DNA Polymerase (Thermo Scientfic)

Each ePCR investigation included the same four sets of samples as Example 2 (A. Normal reaction; B. Polymerase Free (control); C. template free (control); D. daunomycin free (control)).

### Results: Confirmation of Amplification

FIG. 21 shows ePCR performed on screen-printed platinum electrodes. A difference is observed between the ePCR ‘normal reaction’ and the control lacking the polymerase and DM, thus, indicating that amplification of the 40 bp long NMD target was amplified. ePCR-based amplification was obtained by switching the redox-state of DM, through the application of 20 cycles of the reduction/oxidation potentials −1.3 V/−0.2 V, at the working temperature of 75° C. The experiment was only performed once.

A clear difference can be observed between ePCR ‘normal reaction’, which contained all components, compared to the control lacking the polymerase and DM. The higher amount of DNA in the ‘normal reaction’ is most likely an effect of BSA which has prevented DM or polymerase adsorption to the electrode surface. However, it should be noted that the method has only been carried out once so far, thus, no statistical analysis could be performed. Unlike with the screen-printed carbon electrodes, little variation was found in the ‘background’ control samples. Thus, the observed ePCR-based amplification in the ‘normal reaction’ was further substantiated by the low amount of DNA observed in the ‘background’ control samples. Finally, as with the screen-printed carbon electrodes, the difference observed between the ePCR method and ‘conventional PCR’ can be explained by an unoptimised ePCR method.

### REFERENCES

1. Berg, J. M.; Tymaoczko, J. L.; Stryer, L. Biochemistry, 5^{th} ed. W. H. Freeman and Company, New York; 2002.
2. Yakovchuk, P.; Protozanov, E.; Frank-Kamenetskii, M. D. Nucleic Acids Res. 2006, 34, 564-574.
3. Protozanov, E.; Yakovchuk, P.; Frank-Kamenetskii, M. D. J. Mol. Biol. 2004, 342, 775-785.
4. Kool, E. T. Annu. Rev. Biophys. Biomol. Struct. 2001, 30, 1-22.
5. Mergny, J-L.; Lacroix, L. Oligonucleotides 2003, 13, 515-537.
6. Owczarzy, R.; You, Y.; Moreira, B. G.; Manthey, J. A.; Huang, L.; Behlke, M. A.; Walder, J. A. Biochemistry 2004, 43, 3537-3554.
7. Ageno, M.; Dore, E.; Frontali, C. Biophys. J., 1969, 9, 1281-1311.
8. Wang, Y-C.; Lin, C-B.; Su, J-J.; Ru, Y-M.; Wu, Q.; Chen, Z-B.; Mao, B-W.; Tian, Z-W. Anal. Chem. 2011, 83, 4930-4935.
9. Hutton, J. R. Nucleic Acids Res. 1977, 4, 3537-3555.
10. Drummond, T. G.; Hill, M. G.; Barton, J. K. Nat. Biotechnol. 2003, 21, 1192-1199.
11. Bier, F. F.; von Nickisch-Rosenegk, M.; Ehrentreich-Förster, E.; Reiss, E.; Henkel, J.; Strehlow, R.; Andresen, D. Adv. Biochem. Engin./Biotechnol. 2008, 109, 433-453.
12. Leinberger, D. M.; Grimm, V.; Rubtsova, M.; Weile, J.; Schröppel, K.; Thomas, A.; Wichelhaus, T. A.; Knabbe, C.; Schmid, R. D.; Bachmann, T. T. J. Clin. Microbiol. 2010, 48, 460-471.
13. Saiki, R. K.; Gelfand, D. H.; Stoffel, S.; Scharf, S. L.; Higuchi, R.; Horn, G. T.; Mullis, K. B.; Erlich, H. A. Science 1988, 239, 487-491.
14. Craw, P.; Balachandran, W. Lab Chip 2012, 12, 2469-2486.
15. Wiedman, M.; Wilson, W.; Czajka, J.; Luo, J.; Barany, F.; Batt, A. PCR Meth. Appl. 1994, 3, S51-64.
16. Lee, S. J.; Lee, S. Y. Appl. Microbiol. Biotechnol. 2004, 64, 289-299.
17. Asiello, P. J.; Baeumner, A. J. Lab Chip 2011, 11, 1420-1430.
18. Weigl, B.; Domingo, G.; LaBarre, P.; Gerlach, J. Lab Chip 2008, 8, 1999-2014.
19. Holland, C. A.; Kiechle, F. L. Curr. Opin. Microbiol. 2005, 8, 504-509.
20. Yager, P.; Edwards, T.; Helton, K.; Nelson, K.; Tam, M. R.; Weigl, B. H. Nature 2006, 442, 412-418.
21. Luo, X.; Hsing, I-M. Analyst 2009, 134, 1957-1964.
22. Wang, J. Biosens. Bioelectron. 2006, 21, 1887-1892.
23. Asanuma, H.; Liang, X.; Yoshida, T.; Komiyama, M. ChemBioChem 2001, 2, 39-44.
24. Asanuma, H.; Liang, X.; Nishioka, H.; Matsunaga, D.; Liu, M.; Komiyama, M. Nat. Protoc. 2007, 2, 203-212.
25. Ihmels, H.; Otto, D. Topp. Curr. Chem. 2005, 258, 161-204.
26. Long, E. C.; Barton, J. K. Accounts Chem. Res. 1990, 23, 271-273.
27. Erdem, A.; Kerman, K.; Meric, B.; Ozsoz, M. Electroanalysis 2001, 13, 219-223.
28. Fang, T. H.; Ramalingam, N.; Dong, X. D.; Ngin, T. S.; Zeng, X. T.; Kuan, A. T. L.; Huat, E. Y. P.; Gong, H. Q. Biosens. Bioelectron. 2009, 21, 2131-2136.
29. Yeung, S. S. W.; Lee, T. M. H.; Hsing, I-M. Anal. Chem. 2008, 80, 363-368.
30. Deféver, T.; Druent, M.; Evrard, D.; Marchal, D.; Limoges, B. Anal. Chem. 2011, 83, 1815-1821.
31. Quigley, G. J.; Wang, A. H-J.; Ughetto, G.; van der Marel, G.; van Boom, J. H.; Rich, A. Proc. Natl. Acad. Sci. USA 1980, 77, 7204-7208.
32. Chaires, J. B.; Dattagupta, N.; Crothers, D. M. Biochemistryl 982, 21, 3933-3940.
33. Cheng, G.; Qu, H.; Zhang, D.; Zhang, J.; He, P.; Fang, Y. J. Pharma. Biomed. Anal. 2002, 29, 361-369.
34. You, Y.; Tataurov, A. V.; Owczarzy, R. Biopolymers 2011, 95, 472-486.
35. Fredrick, C. A.; Williams, L. D.; Ughetto, G.; van der Marel, G.; van Boom, J. H.; Rich, A.; Wang, A. H-J. Biochemistry 1990, 29, 2538-2549.

1. A method for denaturing or hybridising nucleic acid molecules, said method comprising the steps of contacting a nucleic acid molecule with a compound capable of interacting with a nucleic acid molecule and altering the state or a property of the compound to achieve denaturation or hybridisation of the nucleic acid molecule.

2. The method of claim 1, wherein denaturing nucleic acid molecules comprises de-hybridising, melting or dissociating double stranded nucleic acid molecules to form two separate single strands of nucleic acid.

3. The method of claim 1, wherein hybridising nucleic acid molecules comprises annealing or re-naturing nucleic acid molecules to form a double stranded nucleic acid molecule from two or more single nucleic acid strands.

4. The method of claim 1, wherein the method is used to achieve continual or cyclic denaturation and/or hybridisation of nucleic acids.

5. The method of claim 1, wherein the compound capable of interacting with a nucleic acid molecule interacts by intercalation.

6. The method of claim 1, wherein altering a state or property of the compound capable of interacting with a nucleic acid molecule, comprises altering the oxidation state of said compound.

7. The method of claim 6 wherein altering the oxidation state of the compound is achieved by exposure of the compound to a reducing agent, an oxidising agent and/or electrochemical means.

8. The method of claim 7 wherein the electrochemical means comprises exposing the compound to an electric current or potential.

9. The method of claim 1, wherein the compound capable of interacting with a nucleic acid molecule is daunorubicin and/or a derivative thereof.

10. The method of claim 9, wherein the oxidation state of daunorubicin is altered by electrochemical means.

11. The method of claim 10, wherein the electrochemical means comprises exposing daunorubicin to an electric current or potential to affect denaturation or hybridisation of a nucleic acid molecule.

12. The method of claim 11, wherein exposing daunorubicin to an electric potential in the region of −0.5 V to +0.8 V vs Ag/AgCl, triggers the oxidation of daunorubicin.

13. The method of claim 11, wherein exposing daunorubicin to an electric potential in the region of −1.3 V to −0.3 V vs Ag/AgCl, triggers the reduction of daunorubicin.

14. The method of claim 10, wherein the method exploits an electrochemical apparatus comprising coated electrodes.

15. The method of claim 14, wherein the electrodes are coated with a proteinaceous material.

16. The method of claim 15, wherein the proteinaceous material comprises albumin and/or bovine serum albumin.

17. The method of claim 1, wherein the method is conducted at a working temperature which facilitates the denaturation and/or hybridisation of nucleic acid molecules.

18. The method of claim 1, wherein the method comprises an initial step of determining a working temperature.

19. The method of claim 17, wherein the working temperature is calculated using melting curve analysis.

20. The method of claim 17, wherein the working temperature is calculated by analysing DNA melting at a range of temperatures in the presence of a compound capable of interacting with nucleic acid.

21. The method of claim 19 wherein the melting curve analysis is performed in the presence of oxidised and reduced forms of a compound capable of interacting with nucleic acid.

22. A nucleic acid amplification method wherein the denaturation and/or hybridisation of nucleic acid is achieved by the method of claim 1.

23. The nucleic acid amplification method of claim 22, wherein the amplification method comprises ligase and/or polymerase chain reaction based methods or variations thereof.

24. A kit for hybridising and/or denaturing nucleic acid molecules, said kit comprising:
one or more compounds capable of interacting with nucleic acid molecules; and
a device capable of generating an electric current and/or potential.

25. The kit of claim 24, wherein the kit further comprises components selected from the group consisting of:
(i) buffers, components, reagents, solutions and/or enzymes;
(ii) receptacles and/or tools for manipulating solutions, reagents;
(iii) apparatus for use in an electrochemical method; and
(iv) coating substrate(s) for coating electrodes.

26. The kit of claim 24 or 25, wherein the kit comprises instructions for use.

27. The kit of claim 24, 25 or 26 for use in a method of amplifying or detecting nucleic acid.

28. The kit of claim 27, wherein the kit further comprises one or more components of the polymerase chain reaction.

29. The kit of claim 28, wherein the polymerase chain reaction components are selected from the group consisting of: dNTPs; primers; nucleic acid polymerase; buffer solution(s); and divalent ions.

30. A method of altering the melting temperature of a nucleic acid molecule, said method comprising contacting a nucleic acid molecule with a compound capable of interacting with nucleic acid and, altering a state or property of the compound so as to modulate the melting temperature of the nucleic acid molecule.

31. The method of claim 30, wherein altering a state or property of the compound comprises altering the oxidation status of the compound.

32. The method of claim 30, wherein electrochemical and/or chemical methods are used to alter a state or property of the compound.
